SEQUENCE LISTING (80 sequences)

SEQ ID NO: 1 - protein, 118 aa, synthetic construct
QVQLVQSGAEVKKPGASVKVSCKASGFNIKDFNIHWVRQAPGQGLEWIGRIDPESGDAEYVPKFQGRVTMTRDTSTSTVYMELSSLRSEDTAVYYCTTGSYDLDVEYWGQGTLVTVSS

SEQ ID NO: 2 - protein, 118 aa, synthetic construct
QVQLVQSGAEVKKPGASVKVSCKASGFNIKDFNIHWVRQAPGQGLEWIGRIDPEGGDAEYVPKFQGRVTMTRDTSTSTVYMELSSLRSEDTAVYYCTTGSYDLDVEYWGQGTLVTVSS

SEQ ID NO: 3 - protein, 118 aa, synthetic construct
QVQLVQSGAEVKKPGASVKVSCKASGFNIKDFNIHWVRQAPGQGLEWIGRIDPEQGDAEYVPKFQGRVTMTRDTSTSTVYMELSSLRSEDTAVYYCTTGSYDLDVEYWGQGTLVTVSS

SEQ ID NO: 4 - protein, 118 aa, synthetic construct
QVQLVQSGAEVKKPGASVKVSCKASGFNIKDFNIHWVRQAPGQGLEWIGRIDPEDGDAEYVPKFQGRVTMTRDTSTSTVYMELSSLRSEDTAVYYCTTGSYDLDVEYWGQGTLVTVSS

SEQ ID NO: 5 - protein, 118 aa, synthetic construct
QVQLVQSGAEVKKPGASVKVSCKASGFNIKDFNIHWVRQAPGQGLEWIGRIDPENADAEYVPKFQGRVTMTRDTSTSTVYMELSSLRSEDTAVYYCTTGSYDLDVEYWGQGTLVTVSS

SEQ ID NO: 6 - protein, 118 aa, synthetic construct
QVQLVQSGAEVKKPGASVKVSCKASGFNIKDFNIHWVRQAPGQGLEWIGRIDPENGDAEYVPKFQGRVTMTRDTSTSTVYMELSSLRSEDTAVYYCTTGSYDLDVEYWGQGTLVTVSS

SEQ ID NO: 7 - protein, 8 aa, synthetic construct
FNIKDFNI

SEQ ID NO: 8 - protein, 15 aa, synthetic construct
  VARIANT 8..9: note = SG or GG or QG or DG or NA or NG
IGRIDPEXXDAEYVP

SEQ ID NO: 9 - protein, 10 aa, synthetic construct
TTGSYDLDVE

SEQ ID NO: 10 - protein, 107 aa, synthetic construct
DIQMTQSPSSLSASVGDRVTITCHASQNINVWLSWYQQKPGKAPKLLIYKASTLHTGVPSRFSGSGSGTDFTLTISSLQPEDFATYYCQQGQSYPLTFGQGTKLEIK

SEQ ID NO: 11 - protein, 11 aa, synthetic construct
HASQNINVWLS

SEQ ID NO: 12 - protein, 7 aa, synthetic construct
KASTLHT

SEQ ID NO: 13 - protein, 9 aa, synthetic construct
QQGQSYPLT

SEQ ID NO: 14 - protein, 20 aa, synthetic construct
GGGGSGGGGSGGGGSGGGGS

SEQ ID NO: 15 - protein, 6 aa, synthetic construct
QSISSY

SEQ ID NO: 16 - skipped

SEQ ID NO: 17 - protein, 9 aa, synthetic construct
QQGYPYPFT

SEQ ID NO: 18 - protein, 8 aa, synthetic construct
GFTFSSYA

SEQ ID NO: 19 - protein, 8 aa, synthetic construct
ISGYSGST

SEQ ID NO: 20 - protein, 14 aa, synthetic construct
ARYYSYYGYYYFDY

SEQ ID NO: 21 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQGYPYPFTFGQGTKLEIK

SEQ ID NO: 22 - protein, 121 aa, synthetic construct
EVQLLESGGGLVQPGGSLRLSCAASGFTFSSYAMSWVRQAPGKGLEWVSGISGYSGSTYYADSVKGRFTISRDNSKNTLYLQMNSLRAEDTAVYYCARYYSYYGYYYFDYWGQGTLVTVSS

SEQ ID NO: 23 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQGYPYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 447 aa, synthetic construct
EVQLLESGGGLVQPGGSLRLSCAASGFTFSSYAMSWVRQAPGKGLEWVSGISGYSGSTYYADSVKGRFTISRDNSKNTLYLQMNSLRAEDTAVYYCARYYSYYGYYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 245 aa, Mus musculus
EVRLLQSGAALVRPGASVKLSCTASGFNIKDFNIHWVKQRPEQGLEWIGRIDPENGDAEYVPKFQVRATMTTDTSSNTVYLHLSSLTSGDTAVYYCTTGSYDLDVEYWGQGTTLTVSSGGGGSGGGGSGGGGSGGGGSELQMTQSPSSLSASLGDTVTITCHASQNINVWLSWYQQRPGNIPKLLIYKASTLHTGVPSRFRGSGSGTGFTLTISSLQPEDIATYYCQQGQSYPLTFGAGTKLELK

SEQ ID NO: 26 - protein, 19 aa, synthetic construct
  SITE 19: note = Biotin
FIGITELKKLESKINKVFK

SEQ ID NO: 27 - protein, 16 aa, synthetic construct
FIGITELKKLESKINK

SEQ ID NO: 28 - protein, 15 aa, synthetic construct
FIGITELKKLESKIN

SEQ ID NO: 29 - protein, 14 aa, synthetic construct
FIGITELKKLESKI

SEQ ID NO: 30 - protein, 13 aa, synthetic construct
FIGITELKKLESK

SEQ ID NO: 31 - protein, 12 aa, synthetic construct
FIGITELKKLES

SEQ ID NO: 32 - protein, 32 aa, synthetic construct
LEQLESIINFEKLLAAAAAKFIGITELKKLES

SEQ ID NO: 33 - protein, 32 aa, synthetic construct
FIGITELKKLESLEQLESIINFEKLLAAAAAK

SEQ ID NO: 34 - protein, 12 aa, synthetic construct
  SITE 1: note = Biotin-(PEG3)-
FIGITELKKLES

SEQ ID NO: 35 - protein, 13 aa, synthetic construct
  SITE 12..13: note = (PEG3)
  SITE 13: note = Biotin
FIGITELKKLESK

SEQ ID NO: 36 - protein, 13 aa, synthetic construct
  SITE 13: note = Biotin
FIGITELHHLESK

SEQ ID NO: 37 - protein, 13 aa, synthetic construct
  SITE 13: note = Biotin
FIGISELKKLESK

SEQ ID NO: 38 - protein, 13 aa, synthetic construct
  SITE 13: note = Biotin
FIGITELHKLESK

SEQ ID NO: 39 - protein, 13 aa, synthetic construct
  SITE 13: note = Biotin
FIGITELKHLESK

SEQ ID NO: 40 - protein, 11 aa, synthetic construct
FIGITELKKLE

SEQ ID NO: 41 - protein, 10 aa, synthetic construct
FIGITELKKL

SEQ ID NO: 42 - protein, 9 aa, synthetic construct
FIGITELKK

SEQ ID NO: 43 - protein, 8 aa, synthetic construct
FIGITELK

SEQ ID NO: 44 - protein, 26 aa, synthetic construct
FIGITELLEQLESIINFEKLAAAAAK

SEQ ID NO: 45 - protein, 27 aa, synthetic construct
FIGITELKLEQLESIINFEKLAAAAAK

SEQ ID NO: 46 - protein, 27 aa, synthetic construct
FIGITELHLEQLESIINFEKLAAAAAK

SEQ ID NO: 47 - protein, 12 aa, synthetic construct
ANSKFIGITELK

SEQ ID NO: 48 - protein, 25 aa, synthetic construct
  SITE 1: note = Biotin
IDIKNDLYEKTLNDYKAIANKLSQV

SEQ ID NO: 49 - protein, 245 aa, synthetic construct
QVQLVQSGAEVKKPGASVKVSCKASGFNIKDFNIHWVRQAPGQGLEWIGRIDPESGDAEYVPKFQGRVTMTRDTSTSTVYMELSSLRSEDTAVYYCTTGSYDLDVEYWGQGTLVTVSSGGGGSGGGGSGGGGSGGGGSDIQMTQSPSSLSASVGDRVTITCHASQNINVWLSWYQQKPGKAPKLLIYKASTLHTGVPSRFSGSGSGTDFTLTISSLQPEDFATYYCQQGQSYPLTFGQGTKLEIK

SEQ ID NO: 50 - protein, 245 aa, synthetic construct
QVQLVQSGAEVKKPGASVKVSCKASGFNIKDFNIHWVRQAPGQGLEWIGRIDPEGGDAEYVPKFQGRVTMTRDTSTSTVYMELSSLRSEDTAVYYCTTGSYDLDVEYWGQGTLVTVSSGGGGSGGGGSGGGGSGGGGSDIQMTQSPSSLSASVGDRVTITCHASQNINVWLSWYQQKPGKAPKLLIYKASTLHTGVPSRFSGSGSGTDFTLTISSLQPEDFATYYCQQGQSYPLTFGQGTKLEIK

SEQ ID NO: 51 - protein, 245 aa, synthetic construct
QVQLVQSGAEVKKPGASVKVSCKASGFNIKDFNIHWVRQAPGQGLEWIGRIDPEQGDAEYVPKFQGRVTMTRDTSTSTVYMELSSLRSEDTAVYYCTTGSYDLDVEYWGQGTLVTVSSGGGGSGGGGSGGGGSGGGGSDIQMTQSPSSLSASVGDRVTITCHASQNINVWLSWYQQKPGKAPKLLIYKASTLHTGVPSRFSGSGSGTDFTLTISSLQPEDFATYYCQQGQSYPLTFGQGTKLEIK

SEQ ID NO: 52 - protein, 245 aa, synthetic construct
QVQLVQSGAEVKKPGASVKVSCKASGFNIKDFNIHWVRQAPGQGLEWIGRIDPEDGDAEYVPKFQGRVTMTRDTSTSTVYMELSSLRSEDTAVYYCTTGSYDLDVEYWGQGTLVTVSSGGGGSGGGGSGGGGSGGGGSDIQMTQSPSSLSASVGDRVTITCHASQNINVWLSWYQQKPGKAPKLLIYKASTLHTGVPSRFSGSGSGTDFTLTISSLQPEDFATYYCQQGQSYPLTFGQGTKLEIK

SEQ ID NO: 53 - protein, 245 aa, synthetic construct
QVQLVQSGAEVKKPGASVKVSCKASGFNIKDFNIHWVRQAPGQGLEWIGRIDPENADAEYVPKFQGRVTMTRDTSTSTVYMELSSLRSEDTAVYYCTTGSYDLDVEYWGQGTLVTVSSGGGGSGGGGSGGGGSGGGGSDIQMTQSPSSLSASVGDRVTITCHASQNINVWLSWYQQKPGKAPKLLIYKASTLHTGVPSRFSGSGSGTDFTLTISSLQPEDFATYYCQQGQSYPLTFGQGTKLEIK

SEQ ID NO: 54 - protein, 245 aa, synthetic construct
QVQLVQSGAEVKKPGASVKVSCKASGFNIKDFNIHWVRQAPGQGLEWIGRIDPENGDAEYVPKFQGRVTMTRDTSTSTVYMELSSLRSEDTAVYYCTTGSYDLDVEYWGQGTLVTVSSGGGGSGGGGSGGGGSGGGGSDIQMTQSPSSLSASVGDRVTITCHASQNINVWLSWYQQKPGKAPKLLIYKASTLHTGVPSRFSGSGSGTDFTLTISSLQPEDFATYYCQQGQSYPLTFGQGTKLEIK

SEQ ID NO: 55 - protein, 118 aa, Mus musculus
EVRLLQSGAALVRPGASVKLSCTASGFNIKDFNIHWVKQRPEQGLEWIGRIDPENGDAEYVPKFQVRATMTTDTSSNTVYLHLSSLTSGDTAVYYCTTGSYDLDVEYWGQGTTLTVSS

SEQ ID NO: 56 - protein, 107 aa, Mus musculus
ELQMTQSPSSLSASLGDTVTITCHASQNINVWLSWYQQRPGNIPKLLIYKASTLHTGVPSRFRGSGSGTGFTLTISSLQPEDIATYYCQQGQSYPLTFGAGTKLELK

SEQ ID NO: 57 - protein, 702 aa, synthetic construct
EVQLLESGGGLVQPGGSLRLSCAASGFTFSSYAMSWVRQAPGKGLEWVSGISGYSGSTYYADSVKGRFTISRDNSKNTLYLQMNSLRAEDTAVYYCARYYSYYGYYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSEVRLLQSGAALVRPGASVKLSCTASGFNIKDFNIHWVKQRPEQGLEWIGRIDPENGDAEYVPKFQVRATMTTDTSSNTVYLHLSSLTSGDTAVYYCTTGSYDLDVEYWGQGTTLTVSSGGGGSGGGGSGGGGSGGGGSELQMTQSPSSLSASLGDTVTITCHASQNINVWLSWYQQRPGNIPKLLIYKASTLHTGVPSRFRGSGSGTGFTLTISSLQPEDIATYYCQQGQSYPLTFGAGTKLELK

SEQ ID NO: 58 - protein, 214 aa, synthetic construct; identical to SEQ ID NO: 23

SEQ ID NO: 59 - protein, 702 aa, synthetic construct
EVQLLESGGGLVQPGGSLRLSCAASGFTFSSYAMSWVRQAPGKGLEWVSGISGYSGSTYYADSVKGRFTISRDNSKNTLYLQMNSLRAEDTAVYYCARYYSYYGYYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSQVQLVQSGAEVKKPGASVKVSCKASGFNIKDFNIHWVRQAPGQGLEWIGRIDPENGDAEYVPKFQGRVTMTRDTSTSTVYMELSSLRSEDTAVYYCTTGSYDLDVEYWGQGTLVTVSSGGGGSGGGGSGGGGSGGGGSDIQMTQSPSSLSASVGDRVTITCHASQNINVWLSWYQQKPGKAPKLLIYKASTLHTGVPSRFSGSGSGTDFTLTISSLQPEDFATYYCQQGQSYPLTFGQGTKLEIK

SEQ ID NO: 60 - protein, 702 aa, synthetic construct
EVQLLESGGGLVQPGGSLRLSCAASGFTFSSYAMSWVRQAPGKGLEWVSGISGYSGSTYYADSVKGRFTISRDNSKNTLYLQMNSLRAEDTAVYYCARYYSYYGYYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSQVQLVQSGAEVKKPGASVKVSCKASGFNIKDFNIHWVRQAPGQGLEWIGRIDPESGDAEYVPKFQGRVTMTRDTSTSTVYMELSSLRSEDTAVYYCTTGSYDLDVEYWGQGTLVTVSSGGGGSGGGGSGGGGSGGGGSDIQMTQSPSSLSASVGDRVTITCHASQNINVWLSWYQQKPGKAPKLLIYKASTLHTGVPSRFSGSGSGTDFTLTISSLQPEDFATYYCQQGQSYPLTFGQGTKLEIK

SEQ ID NO: 61 - protein, 214 aa, synthetic construct
DIQMTQSPSSLSASVGDRVTITCRASQSISSYLNWYQQKPGKAPKLLIYAASFLQSGVPSRFSGSGSGTDFTLTISSLQPEDFATYYCQQGYPYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 15 aa, synthetic construct
IGRIDPESGDAEYVP

SEQ ID NO: 63 - protein, 15 aa, synthetic construct
IGRIDPEGGDAEYVP

SEQ ID NO: 64 - protein, 15 aa, synthetic construct
IGRIDPEQGDAEYVP

SEQ ID NO: 65 - protein, 15 aa, synthetic construct
IGRIDPEDGDAEYVP

SEQ ID NO: 66 - protein, 15 aa, synthetic construct
IGRIDPENADAEYVP

SEQ ID NO: 67 - protein, 15 aa, synthetic construct
IGRIDPENGDAEYVP

SEQ ID NO: 68 - protein, 112 aa, synthetic construct
QVQLQQPGAELVMPGASVNLSCKASGYTFTDYWMHWVKQRPGQGLEWIGEIDPSDNFSNLNQNFRGKATLTVDKSSRTAFLQLSSLTSEDSAVYYCAVEDYWGQGTTLTVSS

SEQ ID NO: 69 - protein, 113 aa, synthetic construct
SDIVMTQATPSVLVTPGEAVSISCRASRSLLHSNGITYLYWFLQRPGQSPQVLIYRMSNLVSGVPDRFSGSGSGTAFTLRISRVEAEDVGVYYCMQHLEFPYTFGGGTKLEIK

SEQ ID NO: 70 - protein, 5 aa, synthetic construct
GGGGS

SEQ ID NO: 71 - protein, 10 aa, synthetic construct
GGGGSGGGGS

SEQ ID NO: 72 - protein, 15 aa, synthetic construct
GGGGSGGGGSGGGGS

SEQ ID NO: 73 - protein, 25 aa, synthetic construct
GGGGSGGGGSGGGGSGGGGSGGGGS

SEQ ID NO: 74 - protein, 8 aa, synthetic construct
FIGITELL

SEQ ID NO: 75 - protein, 8 aa, synthetic construct
FIGITELH

SEQ ID NO: 76 - protein, 8 aa, synthetic construct
FIGISELK

SEQ ID NO: 77 - protein, 19 aa, synthetic construct
QAEPDRAHYNIVTFCCKCD

SEQ ID NO: 78 - protein, 28 aa, synthetic construct
FIGITELKKQAEPDRAHYNIVTFCCKCD

SEQ ID NO: 79 - protein, 9 aa, synthetic construct
FIGITELLK

SEQ ID NO: 80 - protein, 9 aa, synthetic construct
FIGITELHK

(19) United States
(12) Patent Application Publication
(10) Pub. No.: US 2026/0199502 A1
(43) Pub. Date: Jul. 16, 2026

(54) BINDING MOLECULE

(71) Applicant: STRIKE PHARMA AB, Uppsala (SE)

(72) Inventors: Helena PERSSON LOTSHOLM, Uppsala (SE); Sara MANGSBO, Uppsala (SE); Oskar ANDERSSON, Uppsala (SE); Johan ROCKBERG, Uppsala (SE); Aman MEBRAHTU, Uppsala (SE)

(21) Appl. No.: 19/136,829

(22) PCT Filed: Dec. 8, 2023
(86) PCT No.: PCT/EP2023/084946

§ 371 Date: Jun. 6, 2025

(30) Foreign Application Priority Data
Dec. 9, 2022 (EP) 22212535.3

(51) Int. Cl. A61K 47/68; A61P 35/00; A61P 37/04; C07K 16/28

(52) U.S. Cl. CPC A61K 47/6811; A61K 47/6889; A61P 35/00; A61P 37/04; C07K 16/2878; C07K 2317/565; C07K 2317/622; C07K 2317/92; C07K 2319/40

(57) ABSTRACT

The present disclosure provides an improved binding molecule comprising an immunoglobulin binding domain, with affinity for a peptide tag moiety. The binding molecule is for example useful as a part of a bispecific conjugate. It exhibits unexpected benefits as part of a biopharmaceutical product, when compared to known binding molecules having a similar binding affinity. Also provided are bispecific conjugates comprising the binding molecule, and complexes comprising the bispecific conjugates non-covalently bound to a tag construct comprising a peptide tag moiety and a cargo moiety which comprises an antigen, for antigen delivery to immune cells. Medical uses of the binding molecules, conjugates and complexes of the disclosure are also provided.

### TECHNICAL FIELD

The present disclosure relates to an improved binding molecule comprising an immunoglobulin binding domain, with affinity for a peptide tag moiety. The binding molecule is for example useful as a part of a bispecific conjugate. It exhibits unexpected benefits as part of a biopharmaceutical product, when compared to known binding molecules having a similar binding affinity. Also provided are bispecific conjugates comprising the binding molecule, and complexes comprising the bispecific conjugates non-covalently bound to a tag construct comprising a peptide tag moiety and an antigen, for antigen delivery to immune cells. Medical uses of the binding molecules, conjugates and complexes of the disclosure are also provided.

### BACKGROUND

Monoclonal antibodies (mAbs) which modulate immune responses are proving highly effective in cancer treatment, with increasing evidence that such responses can be harnessed to provide durable eradication of tumors. Various antibodies against different targets have been developed, e.g. targeting the immune checkpoints CTLA-4 and PD-1, which support the view that T cell immunity can provide an effective treatment for cancer. Promising clinical data have also been obtained with immunostimulatory mAbs that bind agonistically to the co-stimulatory receptor CD40 on antigen-presenting cells (APCs).

Effective stimulation (or priming) of T cells requires not only application of a stimulus to an APC, but also the presentation of antigen by the APC (in the context of an MHC) for recognition and binding by a T cell receptor (TCR). Thus, it is advantageous for the APC to cross-present antigen to T cells for the purpose of T cell stimulation, in other words to take up, process and present antigen (of extracellular origin) to the T cells. However, antigenic material may not always be present (for example if a tumor has been resected, or in the context of a vaccine against an infection), and CD40 agonists may have poor efficacy in driving effective T cell stimulation in such a situation. CD40 stimulation may also be insufficient for T cell activation (for example if there is a dose-limiting toxicity of the CD40 agonist as an infusion product).

For these reasons, it is advantageous to deliver antigen to the APC at the same time as activating CD40 on the APC surface with an agonist. Bispecific conjugates for this purpose are described in WO2020/104690 and WO2021/239968. The bispecific conjugates described therein comprise two binding proteins covalently coupled together. The first binding protein is specific for CD40; the second binding protein, rather than being directly specific for an antigen, is instead specific for a tag moiety. A tag construct is provided, in which the tag moiety is covalently coupled to an antigen, and forms a complex with the bispecific conjugate through the binding affinity of the second binding protein in the conjugate towards the tag moiety. Hence, by binding of the second binding protein to the tag moiety comprised in a tag construct, the bispecific conjugate, comprising the first binding protein covalently coupled to the second binding protein, is bound indirectly to the antigen, providing a flexible, modular approach by which the antigen comprised in the tag construct can be varied, there being no chemical linkage between the antigen and the bispecific conjugate.

WO2020/104690 provides a complex formed between the bispecific conjugate and the tag construct, which complex provides both a CD40 agonist (for activation of an APC) and an antigen (for presentation by the APC). This ensures that APC activation by the CD40 agonist leads to activation of T cells specific for the target antigen, and advantageously allows for flexibility in preparing conjugates and complexes for use in personalized medicine, as well as the use of this flexible platform for vaccine development for vaccination of individuals using the CD40-pathway to mount an effective anti-pathogen immune response.

The complex of WO2020/104690 can also stimulate B cell responses against an antigen. The complex can form two interactions with a B cell: the anti-CD40 binding protein comprised in the bispecific conjugate can bind CD40 on the surface of a B cell and the antigen comprised in the tag construct can bind a specific B cell receptor. The combination of these two interactions activates B cells which recognize the antigen. Indeed, B cells activated in this manner using complexes as described in WO2020/104690 may not require co-stimulation by helper T cells for full activation.

Rather than preparing conjugates comprising an antigen directly fused to a CD40 agonist, or an antigen-specific binder fused to the agonist, which would require the laborious synthesis and production of a separate conjugate for each patient (or at least each different tumor antigen) or for each pathogenic serotype, the bi-specific conjugate can be tailored for individual, personalized use by binding to different tag constructs comprising different antigens but the same tag moiety, according to the need of a particular, individual, patient. The bispecific conjugate is by this strategy tailored to also adapt a personalized or indication-based strategy to vaccinate against a tumor or pathogen with a high antigen drift ensuring a flexible vaccination strategy. In this way, only bespoke tag constructs need to be prepared, providing a benefit in the ease and costs of preparing patient/pathogen-specific therapeutic agents. It is further believed that the indirect and non-covalent binding of antigen to the CD40 agonist may be advantageous for the efficacy of the complex, as compared to a conjugate comprising antigen fused directly and covalently to the CD40 agonist, or as compared to providing the CD40 agonist and antigen separately.

WO2021/239968 discloses a further agonistic antibody (or related binding protein) against CD40, shown to bind CD40 with a high affinity and to display strong agonistic activity. This CD40 binding protein is particularly suited to use in the context of a therapeutic bispecific conjugate, which may similarly be used for cancer therapy or, alternatively, for treatment of or vaccination against an infection.

Together, WO2020/104690 and WO2021/239968 disclose a promising platform for the development of personalized biopharmaceuticals, enabling therapeutic efficacy from CD40 mediated immune activation. However, the development and production of candidate products place high demands on the yield, homogeneity and other expression characteristics of all components or moieties comprised in the product. Also, there is a general need to avoid unwanted immunogenicity in the product in order to enable repeated dosing. While functional and useful, the candidate second binding proteins in the bispecific conjugates disclosed in WO2020/104690 and WO2021/239968, were found to not be optimal in one or more of these considerations, and there is therefore a need in the art to provide improved such binding proteins having a maintained affinity to the tag moiety used in the platform.

### DISCLOSURE OF THE INVENTION

It is an object of the disclosure to meet this need through the provision of a binding molecule which does not exhibit the drawbacks associated with mouse scFv IBIIICI while essentially maintaining the binding characteristics thereof.

It is another object of the disclosure to provide an alternative binding molecule which exhibits a high level of expression when produced in a recombinant expression system.

It is another object of the disclosure to provide an alternative binding molecule which exhibits an increased compatibility with an antibody against e.g. CD40, when used in the context of a bispecific conjugate as described for example in WO2020/104690 and WO2021/239968. An example of increased compatibility is that the binding molecule, when combined with the antibody in a bispecific conjugate, increases the monomeric content achieved upon expression of the bispecific conjugate.

These and other objects which are evident to the skilled person from the present disclosure are met by different aspects of the invention as claimed in the appended claims and as generally disclosed herein.

Thus, in a first aspect, there is provided a binding molecule, comprising:
an immunoglobulin heavy chain variable region (VH) consisting of an amino acid sequence selected from the group consisting of SEQ ID NO:1-6 and amino acid sequences having at least 90% identity thereto, provided that said heavy chain variable region (VH) comprises three complementarity determining domains (CDRs), wherein:
   VHCDR1 has the sequence set forth in SEQ ID NO:7;
   VHCDR2 has the sequence IGRIDPEXaX,DAEYVP (SEQ ID NO:8), wherein XₐX_{b} is selected from the group consisting of SG, GG, QG, DG, NA and NG; and
   VHCDR3 has the sequence set forth in SEQ ID NO:9; and
an immunoglobulin light chain variable region (VL) consisting of an amino acid sequence selected from the group consisting of SEQ ID NO:10 and amino acid sequences having at least 90% identity thereto, provided that said light chain variable region (VL) comprises three complementarity determining domains (CDRs), wherein:
   VLCDR1 has the sequence set forth in SEQ ID NO:11;
   VLCDR2 has the sequence set forth in SEQ ID NO:12; and
   VLCDR3 has the sequence set forth in SEQ ID NO:13.

Although originally conceived as a humanized version of the known mouse antibody IBIIICI as described in Example 1, the binding molecule as defined herein was found to not only exhibit the advantages of having an increased “humanness” score, but also showed unexpected and dramatic benefits over its ancestor when expressed, in terms of for example one or more of its protein expression yield, monomeric content and ease of purification. These advantages were evident both when studying the binding molecule alone, in the scFv format (Example 2, FIG. 2), and when this scFv was placed in the context of a bispecific conjugate similar to those described in WO2020/104690 and WO2021/239968 (Example 6, Table 5, FIGS. 8 and 9). These unexpected improvements in developability parameters are of great importance when developing a commercial biopharmaceutical product.

The increased “humanness” of the binding molecule of the disclosure also makes it less prone to inducing anti-drug antibodies over time when administered to a subject, e.g. a human patient, thus providing benefits that allow for repeated dosing over time. This is advantageous in comparison to non-human proteins as well as to other vector-based platforms which induce anti-drug antibodies, in that such anti-drug antibodies limit the usefulness of the binding molecule over time.

Thus, the present disclosure provides a binding molecule comprising immunoglobulin light and heavy chain variable regions having defined amino acid sequences. The term “binding molecule” is used herein to denote a binding molecule comprising a binding domain of an antibody (that is to say, a binding domain obtained or derived from an antibody, or based on a binding domain of an antibody). Thus, the binding molecule is an antibody-based, or antibody-like, molecule comprising the binding site of, or a binding site derived from, an antibody.

As noted above, a binding domain of an antibody is composed of a light chain variable domain and a heavy chain variable domain (thus a classical bivalent antibody has two binding domains). A binding molecule may thus be a native antibody or a fragment thereof, or an artificial or synthetic antibody, or an antibody construct, or derivative (e.g. a single chain antibody). In summary, the binding molecule of the disclosure comprises a binding domain of an antibody, said binding domain of an antibody comprising a light chain variable domain and a heavy chain variable domain.

As detailed above, the binding molecule of the disclosure comprises six CDR sequences. The light and heavy chain variable domains comprise 3 CDRs each: the heavy chain variable domain comprises VHCDR1, VHCDR2 and VHCDR3, and the light chain variable domain comprises VLCDR1, VLCDR2 and VLCDR3. The six CDRs have the following amino acid sequences:
VHCDR1 has the sequence set forth in SEQ ID NO:7;
VHCDR2 has the sequence set forth in SEQ ID NO:8;
VHCDR3 has the sequence set forth in SEQ ID NO:9;
VLCDR1 has the sequence set forth in SEQ ID NO:11;
VLCDR2 has the sequence set forth in SEQ ID NO:12; and
VLCDR3 has the sequence set forth in SEQ ID NO:13.

As described above, SEQ ID NO:8 is the amino acid sequence IGRIDPEXₐX_{b}DAEYVP, wherein the amino acid pair XₐX_{b} is selected from the group consisting of SG, GG, QG, DG, NA and NG. In other words, the sequence of the VHCDR2 of the binding molecule's heavy chain variable domain is selected from the group consisting of SEQ ID NO:62-67. As shown in the Example section herein, XₐX_{b} can be varied in this way without substantially affecting the binding of the binding molecule to the amino acid sequence of its tag moiety counterpart. In individual embodiments of the binding molecule of the disclosure, the six different versions of the VHCDR2 sequence are combined into subgroups comprising every possible sub-combination of 2, 3, 4 and 5 different VHCDR2 sequences. In even more specific embodiments, each individual VHCDR2 sequence represents an individual embodiment of the binding protein. The resulting disclosure of all possible combinations of SEQ ID NO:1-6 (which is equivalent to all possible combinations of SEQ ID NO:62-67) is given below in the ITEMIZED LISTING OF EMBODIMENTS, items 2-63. All such combinations are, each individually, part of the disclosure herein.

The binding molecule of the disclosure comprises a heavy chain variable domain having a sequence selected from SEQ ID NO:1-6 and any sequence having at least 90% identity to any one of SEQ ID NO:1-6, as long as the three CDR sequences consist of SEQ ID NO:7-9. In specific embodiments, the sequence has at least 91%, such as at least 92%, such as at least 93%, such as at least 94%, such as at least 95%, such as at least 96%, such as at least 97%, such as at least 98%, such as at least 99% identity to any one of SEQ ID NO:1-6.

The binding molecule of the disclosure comprises a light chain variable domain having the sequence SEQ ID NO:10 and any sequence having at least 90% identity to SEQ ID NO:10, as long as the three CDR sequences consist of SEQ ID NO: 11-13. In specific embodiments, the sequence has at least 91%, such as at least 92%, such as at least 93%, such as at least 94%, such as at least 95%, such as at least 96%, such as at least 97%, such as at least 98%, such as at least 99% identity to SEQ ID NO:10.

As described herein, binding molecules having amino acid sequences with 90% or more identity to the listed sequences are also contemplated to fall within the ambit of the disclosure. Such variant sequences may be modified relative to the listed sequences by substitution, insertion and/or deletion of one or more amino acids.

An amino acid substitution relative to a listed sequence may be a conservative amino acid substitution. The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus a conservative amino acid substitution may be considered to be a substitution in which a particular amino acid residue is substituted for a different amino acid in the same family. However, an amino acid substitution may equally be a non-conservative substitution, in which one amino acid is substituted for another with a side-chain belonging to a different family.

As detailed above, according to the present disclosure variants of the listed sequences have at least 90% sequence identity to the respective listed sequence. Sequence identity may be assessed by any convenient method. However, for determining the degree of sequence identity between sequences, computer programs that make pairwise or multiple alignments of sequences are useful, for instance EMBOSS Needle or EMBOSS stretcher (both Rice et al. (2000), Trends Genet., 16(6):276-277) may be used for pairwise sequence alignments, while Clustal Omega (Sievers et al. (2011), Mol. Syst. Biol. 7:539) or MUSCLE (Edgar (2004), Nucleic Acids Res. 32(5):1792-1797) may be used for multiple sequence alignments, though any other appropriate program may be used. Whether the alignment is pairwise or multiple, it must be performed globally (i.e. across the entirety of the reference sequence) rather than locally.

Sequence alignments and % identity calculations may be determined using for instance standard Clustal Omega parameters: matrix Gonnet, gap opening penalty 6, gap extension penalty 1. Alternatively the standard EMBOSS Needle parameters may be used: matrix BLOSUM62, gap opening penalty 10, gap extension penalty 0.5. Any other suitable parameters may alternatively be used.

The binding molecule of the disclosure may be synthesized by any method known in the art. Preferably, the binding molecule is synthesized using a protein expression system, such as a cellular expression system using prokaryotic (e.g. bacterial) cells or eukaryotic (e.g. yeast, fungus, insect or mammalian) cells. An alternative protein expression system is a cell-free, in vitro expression system, in which a nucleotide sequence encoding the binding molecule is transcribed into mRNA, and the mRNA translated into a protein, in vitro. Cell-free expression system kits are widely available, and can be purchased from e.g. ThermoFisher. Alternatively, binding proteins may be chemically synthesized in a non-biological system. Liquid-phase synthesis or solid-phase synthesis may be used to generate polypeptides which may form or be comprised within the binding molecule of the disclosure.

The skilled person can readily produce binding proteins using appropriate methodology common in the art. In particular, the binding molecule may be recombinantly expressed in mammalian cells, such as CHO cells. A binding molecule synthesized in a protein expression system may be purified using standard techniques in the art, e.g. it may be synthesized with an affinity tag and purified by affinity chromatography. If the binding molecule is an antibody, it can be purified using affinity chromatography using one or more antibody-binding proteins, such as Protein G, Protein A, Protein A/G or Protein L.

As noted above, the binding molecule is an antibody-based, or antibody-like, molecule. Thus, a binding molecule may be a native antibody or a fragment thereof, or an artificial or synthetic antibody, or an antibody construct or derivative (e.g. a single chain antibody).

In one embodiment, the binding molecule is an antibody, in particular a monoclonal antibody. By “monoclonal antibody” is meant an antibody preparation consisting of a single antibody species, i.e. all antibodies in the preparation have the same amino acid sequences, including the same CDRs, and thus bind the same epitope on their target antigen (by “target antigen” is meant the antigen containing the epitope bound by a particular antibody) with the same effect. In other words, the antibody of the disclosure is preferably not part of a polyclonal mix of antibodies.

If the binding molecule is an antibody, the antibody may be of any isotype and subtype. Thus it may be an IgA, IgD, IgE, IgG, or IgM antibody. The heavy-chain constant domains that correspond to the different isotypes of immunoglobulins are termed α, δ, ε, γ and μ, respectively. The subunit structures and three-dimensional configurations of different isotypes of immunoglobulins are well known. Preferably the antibody is an IgG antibody. As noted above, there are four subtypes of IgG antibody: IgG1, IgG2, IgG3 and IgG4. The IgG anti-CD40 antibody of the disclosure may be of any IgG subtype, i.e. it may be an IgG1, IgG2, IgG3 or IgG4 antibody.

In one embodiment, the binding molecule is a binding fragment of an antibody (i.e. an antibody fragment), that is a fragment which retains the ability of the antibody to bind specifically to its target antigen. Such fragments are well known and examples include Fab′, Fab, F(ab′)₂, Fv, Fd, or dAb fragments, which may be prepared according to techniques well known in the art.

A Fab fragment consists of the antigen binding domain of an antibody, i.e. an individual antibody may be seen to contain two Fab fragments, each consisting of a light chain and its conjoined N-terminal section of a heavy chain. Thus a Fab fragment contains an entire light chain and the VH and CH1 domains of the heavy chain to which it is bound. Fab fragments may be obtained by digesting an antibody with papain.

F(ab′)₂ fragments consist of the two Fab fragments of an antibody, plus the hinge regions of the heavy domains, including the disulphide bonds linking the two heavy chains together. In other words, a F(ab′)₂ fragment can be seen as two covalently joined Fab fragments. F(ab′)₂ fragments may be obtained by digesting an antibody with pepsin. Reduction of F(ab′)₂ fragments yields two Fab′ fragments, which can be seen as Fab fragments containing an additional sulfhydryl group which can be useful for conjugation of the fragment to other molecules.

In an alternative and preferred embodiment, the binding molecule is a synthetic or artificial construct, i.e. an antibody-like molecule which comprises a binding domain, but which is genetically engineered or artificially constructed. Such constructs include chimeric or CDR-grafted antibodies, as well as single chain antibodies and other constructs, e.g. scFv, dsFv, ds-scFv, dimers, minibodies, diabodies, single domain antibodies (DABs), TandAbs dimers and heavy chain antibodies such as VHH, etc. In a particular embodiment, the binding molecule is a single chain variable fragment (scFv). An scFv is a fusion protein in which a single polypeptide comprises both the VH and VL domains of an antibody. scFv fragments generally include a peptide linker covalently joining the VH and VL regions, which contributes to the stability of the molecule. The linker may comprise from 1 to 20 amino acids, such as for example 1, 2, 3 or 4 amino acids, 5, 10 or 15 amino acids, or other intermediate numbers in the range 1 to 20 as convenient. The peptide linker may be formed from any generally convenient amino acid residues, such as glycine and/or serine. One example of a suitable linker is G₄S (SEQ ID NO:70). Multimers of such linkers may be used, such as for example a dimer, a trimer, a tetramer or a pentamer, i.e. (G₄S)₂ (SEQ ID NO:71), (G₄S)₃ (SEQ ID NO:72), (G₄S)₄ (SEQ ID NO: 14) or (G₄S)₅ (SEQ ID NO:73). In a particular embodiment in which the binding molecule of the disclosure is an scFv, the linker is a (G₄S)₄ linker (SEQ ID NO:14). However, it is not essential that a linker be present, and the VL domain may instead be linked directly to the VH domain by a peptide bond. An scFv typically comprises, N-terminal to C-terminal, a VH region linked to a VL region by a linker sequence. In other words, in such an embodiment of a binding molecule of the disclosure, the scFv comprises a peptide linker joining the C terminus of the heavy chain variable region (VH) to the N terminus of the light chain variable region (VL).

The preparation of scFv molecules is well known in the art, and a person of ordinary skill is able to readily prepare scFv binding molecules according to the disclosure which comprise the VH and VL sequences defined herein. Thus, by way of non-limiting example, the binding molecule of the disclosure may be an scFv comprising an amino acid sequence selected from the group consisting of SEQ ID NO: 49-54 and sequences having at least 90, 91, 92, 93, 94, 95, 96, 97, 98 or 99% identity thereto. The disclosure also contemplates any individual scFv sequence or subgroup of sequences, in analogy to the listing in the “ITEMIZED LISTING OF EMBODIMENTS” of individual pairs of VH and VL sequences (see items 2-63). In a particular embodiment, the binding molecule of the disclosure is an scFv comprising the amino acid sequence SEQ ID NO:49, denoted “SG” or “IBIIICI CDR graft SG”.

In one embodiment, the binding molecule is a humanized protein, in particular a humanized scFv. A humanized binding molecule may comprise VH and VL regions in which framework are derived from human germline immunoglobulin sequences. Such proteins may however include amino acids not encoded by human germline Ig sequences, for example mutations introduced by random or site-specific mutagenesis, or amino acids derived from ancestral binding molecules generated in other species, e.g. a mouse.

In one embodiment of the binding molecule of the disclosure, it is capable of selective binding to a peptidic tag moiety comprised in a tag construct. By “selective binding” is meant that the binding molecule binds to its target in a manner that can be distinguished from binding to non-target molecules, more particularly that the binding molecule binds its target with greater binding affinity than with which it binds other molecules. That is, the binding molecule does not bind to other, non-target, molecules, or does not do so to an appreciable or significant degree, or binds with lower affinity to such other molecules than with which it binds the tag moiety. A binding molecule “that selectively binds” the tag moiety may alternatively be referred to as “directed against” or “recognizing” the tag moiety. In other words, the tag moiety is the antigen of the binding molecule of the disclosure, and the binding molecule is thus an “antigen binding protein” in the sense that it binds the tag moiety as its antigen. The skilled person will realize that the terms “antigen” and “antigen binding protein” in the previous sentence are applied in the context of a metaphor in an attempt to illustrate the binding relationship between the tag moiety and the binding molecule of the disclosure. The skilled person will also realize that the previous sentence is not indicative of the tag moiety being an antigen itself which induces an unspecific or specific immune reaction towards it, but rather that the tag moiety is a bespoke binding partner to the binding molecule of the disclosure.

In one embodiment of the disclosure, the tag moiety, to which the binding molecule is capable of specific binding, comprises the amino acid sequence FIGITELK (SEQ ID NO:43). Examples of such tag moieties, comprising SEQ ID NO: 43, are tested in the experimental section below and form the group consisting of SEQ ID NO:26-35, 39-43, 45 and 47. A particular tag moiety to which the binding molecule herein is capable of specific binding consists of SEQ ID NO:42, i.e. FIGITELKK.

In another embodiment, the tag moiety comprises the amino acid sequence FIGITELL (SEQ ID NO:74), for example selected from the group consisting of SEQ ID NO:44 and 74. In another embodiment, the tag moiety comprises the amino acid sequence FIGITELH (SEQ ID NO:75), for example selected from the group consisting of SEQ ID NO:36, 38, 46 and 75. In another embodiment, the tag moiety comprises the amino acid sequence FIGISELK (SEQ ID NO:76), for example selected from the group consisting of SEQ ID NO:37 and 76. In one embodiment, the tag moiety comprises the amino acid sequence FIGITELLK (SEQ ID NO:79). the tag moiety comprises the amino acid sequence FIGITELHK (SEQ ID NO:80).

As established in WO2020/104690 and WO2021/239968, the tag moiety to which the second binding protein binds can advantageously form part of a tag construct. In one embodiment, the tag construct comprising the peptide tag moiety further comprises at least one cargo moiety. Said at least one cargo moiety may for example be an antigen moiety, to which it may be of interest to elicit an immune response. Said at least one cargo moiety may for example be a peptidic moiety, such as an antigen peptidic moiety. For clarity, the “antigen moiety” (an example of a type of “cargo moiety”) present in one embodiment of a tag construct is different from the “tag moiety” comprised in said construct, notwithstanding the fact that the tag moiety may be considered to be the antigen to which the binding molecule of the disclosure is capable of selective binding. In embodiments wherein said tag construct comprises a peptide tag moiety and a peptide cargo moiety (e.g. a peptidic antigen or an “antigen moiety”), the C terminus of said tag moiety may be covalently bound to the N terminus of said cargo moiety, or vice versa. In another embodiment, a cargo moiety is a nucleic acid moiety.

In embodiments wherein the at least one cargo moiety is a nucleic acid moiety, the tag moiety and cargo moiety can be arranged in either order in a polypeptide-nucleic acid combination. In one such embodiment, the cargo moiety is an siRNA molecule.

In one embodiment of the binding molecule of the present disclosure, it is capable of binding to the tag moiety such that the K_{D} value of the interaction is at most 5×10⁻⁹ M, for example at most 1×10⁻¹⁰ M, for example at most 1×10⁻¹¹ M. The skilled person will understand that the binding interaction is between the binding protein of the present disclosure and the tag moiety.

In a second aspect, the disclosure provides a bispecific conjugate comprising
at least one first moiety which is a binding molecule of the disclosure; and
at least one second moiety which is an antibody or antigen binding fragment thereof.

The design considerations for creating a bispecific conjugate of this aspect of the disclosure are analogous to what is described for similar conjugates in WO2020/104690 and WO2021/239968. Herein, however, the first moiety of the bispecific conjugate is a binding molecule according to the first aspect, which, in one embodiment, is advantageously capable of binding to a tag moiety. In one embodiment herein, the second moiety is an antibody or antigen binding fragment thereof with a binding affinity for an immunologically relevant target.

In one embodiment of the bispecific conjugate of the disclosure, said second moiety is an antigen binding fragment of an antibody, said fragment being selected from the group consisting of a Fab fragment, a Fab′ fragment, a F(ab′)₂ fragment, an Fc fragment, an Fv fragment, a single chain (scFv) fragment, an (scFv)₂ and a domain antibody.

In one embodiment of the bispecific conjugate, said second moiety is an antibody, optionally of the IgG2 subtype.

In one embodiment of the bispecific conjugate, said first moiety is covalently linked to said second moiety, optionally via a linker peptide, for example a linker selected from the linkers discussed above in connection with the first aspect. Thus, if present, the linker may comprise from 1 to 20 amino acids, and may for example be formed from any generally convenient amino acid residues, such as glycine and/or serine. One example of a suitable linker is G₄S (SEQ ID NO:70) and multimers thereof such as (G₄S)₂ (SEQ ID NO:71), (G₄S)₃ (SEQ ID NO:72), (G₄S)₄ (SEQ ID NO: 14) or (G₄S)₅ (SEQ ID NO:73).

In one embodiment of the bispecific conjugate, said first moiety is covalently linked to said second moiety via a linker peptide.

In an alternative embodiment of the bispecific conjugate, said first moiety is linked to said second moiety directly.

In one embodiment of the bispecific conjugate, said first moiety is covalently linked (directly or via a linker) to a C- or N-terminal end of a light or heavy chain of said second moiety.

In one embodiment of the bispecific conjugate, it comprises two first moieties and one antibody as second moiety, and one first moiety is conjugated to the CH3 domain of each heavy chain of said second moiety antibody.

In another embodiment of the bispecific conjugate, it comprises two first moieties and one antibody as second moiety, and one first moiety is conjugated to the CL domain of each light chain of said second moiety antibody.

In a particularly interesting embodiment of the bispecific conjugate, said second moiety is an anti-CD40 antibody or antigen binding fragment thereof.

In such an embodiment, the second moiety may for example be selected from the group consisting of CP-870,893, APX005M, ADC-1013, ChiLob 7/4, SEA-CD40 and ABS-1150/1151 and an antibody comprising an antigen binding fragment derived from any one or more of said antibodies.

Alternatively, the second moiety can be an anti-CD40 binding antibody or antigen binding fragment thereof as described in WO2021/239968, for example the antibody designated “A9” therein. In one such embodiment, the second moiety is an anti-CD40 antibody or antigen binding fragment thereof comprising the following six CDRs: VLCDR1: SEQ ID NO:15; VLCDR2: SEQ ID NO:16; VLCDR3: SEQ ID NO: 17; VHCDR1: SEQ ID NO:18; VHCDR2: SEQ ID NO:19; and VHCDR3: SEQ ID NO: 20. In a more specific such embodiment, the light chain variable domain (VL) of said anti-CD40 antibody comprises an amino acid sequence selected from SEQ ID NO: 21 and amino acid sequences having at least 90% sequence identity thereto; and the heavy chain variable domain (VH) of said anti-CD40 antibody comprises an amino acid sequence selected from SEQ ID NO:22 and amino acid sequences having at least 90% sequence identity thereto. For example, such an anti-CD40 antibody may comprise a light chain (LC) comprising an amino acid sequence selected from SEQ ID NO:23 and amino acid sequences having at least 90% sequence identity thereto; and a heavy chain (HC) comprising an amino acid sequence selected from SEQ ID NO:24 and amino acid sequences having at least 90% sequence identity thereto. In analogy to the sequences disclosed for the binding molecule according to the first aspect of the disclosure, in specific embodiments, a VH, VL, HC or LC sequence of the anti-CD40 antibody may have at least 91%, such as at least 92%, such as at least 93%, such as at least 94%, such as at least 95%, such as at least 96%, such as at least 97%, such as at least 98%, such as at least 99% identity to either one of the respective listed sequences SEQ ID NO:21-24.

Examples of individual bispecific conjugates of this aspect, which have been tested in the experimental section herein and found to exhibit desirable characteristics, are those denoted SP007, SP019 and SP027 herein. Each of these bispecific conjugates consist of two copies of a polypeptide chain comprising a first moiety which is an scFv embodiment of the binding molecule of the first aspect and an antibody heavy chain of a certain anti-CD40 antibody, and two copies of a polypeptide chain which is an antibody light chain of the same anti-CD40 antibody (see Table 4 in Example 6).

In a third aspect of the disclosure, there is provided a polynucleotide encoding a binding molecule or a bispecific conjugate as described herein; an expression vector comprising said polynucleotide; and a host cell comprising said expression vector. Also encompassed by this disclosure is a method of producing a binding molecule or bispecific conjugate of the disclosure, comprising culturing said host cell under conditions allowing expression of said binding molecule or bispecific conjugate from its expression vector, and isolating the binding molecule or bispecific conjugate.

In a fourth aspect, the disclosure provides a complex, such as a non-covalent complex, comprising a bispecific conjugate of the disclosure and a tag construct of the disclosure comprising a tag moiety, to which the binding molecule of the first aspect is capable of binding. In embodiments of the complex, the tag construct is as defined above when discussed in connection with the first aspect of the disclosure. A complex of this aspect may be formed by contacting the bispecific conjugate with the tag construct, such that the tag construct is bound by the binding molecule of the conjugate, through the binding molecule's affinity for the tag moiety comprised in the tag construct.

In a further aspect, the disclosure provides a pharmaceutical composition comprising (i) a binding molecule of the disclosure, as described above; (ii) a bispecific conjugate of the disclosure, as described above, or (iii) a complex of the disclosure, as described above. In addition to the binding protein, bispecific conjugate or complex, the pharmaceutical composition also comprises at least one pharmaceutically acceptable carrier or excipient.

Also provided by the present disclosure are kits and products comprising, separately, a bispecific conjugate and a tag construct as described above. In such kits and products the conjugate and tag construct may be separately provided in compositions containing a pharmaceutically acceptable carrier or excipient.

As used herein, “pharmaceutically acceptable carrier or excipient” includes any and all solvents, dispersion media, coatings, antibacterial and antifungal agents, isotonic and absorption delaying agents and the like that are physiologically compatible.

Preferably, the carrier or excipient is suitable for parenteral, e.g. intradermal, intravenous, intramuscular or subcutaneous administration (e.g. by injection or infusion). Depending on the route of administration, the binding protein, bispecific conjugate, complex or constituent component thereof may be coated in a material to protect the it from the action of acids and other natural conditions that may inactivate or denature it.

Preferred pharmaceutically acceptable carriers comprise aqueous carriers or diluents. Examples of suitable aqueous carriers that may be employed in the pharmaceutical compositions, kits and products include water, buffered water and saline. Examples of other carriers include ethanol, polyols (such as glycerol, propylene glycol, polyethylene glycol, and the like), and suitable mixtures thereof, vegetable oils and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, sodium chloride and the like.

The pharmaceutical composition, product or kit also may include a pharmaceutically acceptable anti-oxidant. They may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol sorbic acid, and the like. In addition, prolonged absorption of the injectable pharmaceutical form may be brought about by the inclusion of agents which delay absorption such as aluminum monostearate and gelatin.

Therapeutic compositions typically must be sterile and stable under the conditions of manufacture and storage. The composition can be formulated as a solution, microemulsion, liposome, or other ordered structure suitable to high drug concentration.

Sterile injectable solutions can be prepared by incorporating the active agent (e.g. complex) in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

Pharmaceutical compositions, products and kits may comprise additional active ingredients as well as the binding protein, bispecific conjugate, complex or component thereof, for example they may comprise additional therapeutic or prophylactic agents. Thus, the complex may be used as a monotherapy or as part of a combination therapy, e.g. in the treatment of cancer. A kit or combination product as described herein may additionally contain instructions for use.

The binding protein of the present disclosure, bispecific conjugate of the present disclosure, complex of the present disclosure, pharmaceutical composition of the present disclosure, kit of the present disclosure and combination product of the present disclosure may be used in therapy. The disclosure thus provides the binding protein, bispecific conjugate, complex, pharmaceutical composition or kit of the disclosure for use in therapy. By therapy is meant the treatment of a subject. By “therapy” as used herein is meant the treatment of any medical condition. Such treatment may be prophylactic (i.e. preventative), curative (or treatment intended to be curative), or palliative (i.e. treatment designed merely to limit, relieve or improve the symptoms of a condition). In curative and palliative applications, complexes or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”. Effective amounts for a given purpose will depend on the disease or condition to be treated, its severity and the size/weight and general state of the subject.

Prophylactic treatment may include the prevention of a condition, or a delay in the development or onset of a condition. For example the complex may be used to prevent an infection, or to reduce the extent to which an infection may develop, or to prevent, delay or reduce the extent of a cancer developing, or recurring, or for example to prevent or reduce the extent of metastasis.

A subject, as defined herein, refers to any mammal, e.g. a farm animal such as a cow, horse, sheep, pig or goat, a pet animal such as a rabbit, cat or dog, or a primate such as a monkey, chimpanzee, gorilla or human. Most preferably the subject is a human.

The combination product of the disclosure comprises a bispecific conjugate as defined herein and a tag construct as defined herein as a combined preparation for simultaneous or sequential use in therapy. That is to say, when the combination product disclosed herein is used according to the disclosure, i.e. in therapy, the conjugate and tag construct are administered simultaneously or sequentially to the subject. Similarly, when the kit of the disclosure is used in therapy, the bispecific conjugate and tag construct are administered simultaneously or sequentially to the subject. By “simultaneous” administration, as used herein, means that the two components are administered to the subject at the same time, or at least substantially the same time, by the same administrative route and at substantially the same location. By “sequential” administration, as used herein, is meant that the two components are administered to the subject at different times. In particular, administration of the first component is completed before administration of the second component commences.

Due to the nature of the present disclosure, sequential administration of the bispecific conjugate and tag construct requires both to be administered by the same route and at substantially the same location. Furthermore, although the administration of the conjugate and tag construct may be temporally spaced, the interval between the administrations should be such as to allow a complex to be formed, when both components have been administered. Thus, for example, both components may be administered within 1 hour of each other, or more particularly within 40, 30, 20, 15, 10, 8, 7, 6, 5, 4, 3, 2 or 1 minute, or less than a minute, of each other.

Mention of the complex of the disclosure (or a pharmaceutical composition comprising such a complex) being administered to a subject for therapeutic purposes is to be understood as referring to a pre-mixed composition (e.g. solution) comprising both components of the complex (i.e. a bispecific conjugate of the disclosure and a tag construct as described above), which components may exist in a dynamic equilibrium comprising the complex and its two individual components.

In particular, the binding protein, bispecific conjugate, complex, composition, kit or combined product of the disclosure may be used in the treatment or prevention of cancer. By cancer is meant any malignant or pre-malignant neoplastic condition. The cancer may be thus be any cancer, of any organ, tissue or cell type. Cancers which present as solid tumors, and which do not exhibit solid tumors are included. Accordingly, haemopoietic cancers are included.

The cancer may be prostate cancer, breast cancer, colorectal cancer, pancreatic cancer, ovarian cancer, lung cancer, cervical cancer, rhabdomyosarcoma, neuroblastoma, multiple myeloma, leukemia, acute lymphoblastic leukemia, melanoma, bladder cancer, head and neck cancer, lymphoma, glioblastoma, or skin cancer. It may also be adrenal cancer, bone cancer, brain cancer, esophageal cancer, eye cancer, gastric cancer, oral cancer, penile cancer, testicular cancer, thyroid cancer, uterine cancer, and vaginal cancer. Mast cell tumors and hemangiosarcoma may also be treated according to the present disclosure. The cancer may be newly diagnosed and naïve to treatment or it may be relapsed or refractory, or relapsed and refractory, primary or metastatic.

When present in a bispecific conjugate of the disclosure, a CD40-specific second moiety activates the immune system by agonizing or stimulating CD40 on APCs, particularly on dendritic cells. In particular, this may lead to T cell activation. The subsequent immune response exerts an anti-cancer effect on neighboring or accessible tumor cells, without regard to CD40 expression by the tumor. A bispecific conjugate of this embodiment, or complex containing it, may therefore be effective against both CD40-positive and CD40-negative cancers. Such a bispecific conjugate can also exert its effect as an adjuvant within a vaccination regimen against a pathogen. If a vaccine platform (e.g. an attenuated virus or a DNA/RNA-based vaccine) does not stimulate a sufficient immune response by itself, CD40 activation may be required to stimulate effective anti-pathogen immune responses resulting in neutralizing antibodies or T cell responses.

In addition to the agonistic immune activating effect provided by the CD40-specific second moiety in embodiments wherein it is present, the complex of the disclosure also provides an antigen, which may be processed and presented to T cells which are activated, and thus T cells may be primed to target cancer cells which express the antigen or to a virally infected cell. This may be of particular benefit in circumstances where cancer antigen presence is low or reduced, for example where a tumor has been surgically removed, in cases where an anti-CD40 therapeutic cannot be delivered intra-tumorally, or when antigen presentation is not ideally performed at the tumor site due to suppressive factors secreted by the tumor. The complex according to this embodiment provides a means for providing cancer antigen in the vicinity of the agonistic activation signal, e.g. at a non-tumor site to ensure T cell priming and activation that can later travel to the tumor and exert their function.

In some embodiments, the cancer antigen to be delivered by the complex may be selected based on the subject and the particular cancer, thus allowing personalized medicine. For example, the cancer of the subject may be subjected to genetic profiling, allowing a suitable antigen to be selected. A bank or library of antigens, or tag construct(s) comprising antigens in the form of cargo moieties, may be provided from which a suitable tag construct may be prepared or selected depending on the cancer type of the subject.

The bispecific conjugate and complex (and kit and combination product) of the disclosure may also be useful in the treatment or prevention of an infection. The present disclosure may in particular be used in therapy for (e.g. vaccination against) viral infections, in particular infections caused by RNA viruses. Infections caused by RNA viruses which may be treated of prevented (by vaccination) according to the present disclosure include infections caused by coronaviruses (such as SARS-COV 1 (the causative agent of SARS), SARS COV 2 (the causative agent of COVID 19) and MERS-COV), influenza viruses, ebola virus, hepatitis C virus (HCV), hepatitis E virus (HEV), rabies virus, poliovirus, Ross River virus and measles virus.

The disclosure may also be used in treatment for or vaccination against infections caused by Epstein-Barr virus (EBV), cytomegalovirus (CMV), human herpes viruses (e.g. HHV 6), parvovirus B19 and human papillomavirus (HPV), though any viral infection can, in principle, be treated or prevented according to the present disclosure.

Intracellular bacterial infections may also be treated according to the present disclosure, e.g. Brucellosis (caused by bacterial species of the genus Brucella), Q fever (caused by Coxiella burnetii), diseases caused by species of Chlamydiae, such as chlamydia (caused by Chlamydia trachomatis) and pneumonia (caused by Chlamydia pneumoniae), leprosy (caused by Mycobacterium leprae and Mycobacterium lepromatosis) and tuberculosis, including disseminated tuberculosis (caused by Mycobacterium tuberculosis). Intracellular fungal or protozoal infections may also be treated by the current disclosure, including leishmaniasis (caused by trypanosomes of the genus Leishmania) and toxoplasmosis (caused by the apicomplexan Toxoplasma gondii). The antigen may thus be derived from any of the aforementioned pathogens.

In the case of a bispecific conjugate comprising an anti-CD40 antibody or fragment thereof, agonism of CD40 may activate the immune system to fight the infection (on a similar principle to use in therapy for cancer). In the case of the complex of the disclosure, as described above this may be provided with an antigen derived from the target pathogen, thus inducing a specific immune response against the pathogen. The vaccine platform is beneficially adaptable for e.g. a pandemic situation. This adaptability can in particular be provided by using a tag construct comprising at least one cargo moiety which is an antigen that can be modified for viral diversity and antigen drift. Viral antigens can be selected based on HLA prevalence in a certain region along with viral serotype determinants.

The bispecific conjugate of the disclosure may, in one embodiment, comprise an antagonistic anti-CD40 antibody or antigen binding fragment thereof. In such an embodiment, the bispecific conjugate is useful to tolerize the immune system against a target that drives an autoimmune disease. In one embodiment of the complex of the disclosure, it comprises a bispecific conjugate in which the second moiety is an antagonist of CD40 and a tag construct comprising at least one cargo moiety which is an antigen known to be the driver for an autoimmune disease, such as an autoimmune disease caused by T cells. The purpose of the bispecific conjugate in complex with the tag construct comprising at least one cargo moiety which is an antigen in this case is to re-educate the immune system to tolerize against the antigen causing disease, and it may thereby prevent the excessive destruction of healthy tissue. Examples of antigens useful in tag constructs according to such an embodiment are selected from the group consisting of antigens associated with SLE, type I diabetes, rheumatoid arthritis, vasculitis, myositis, multiple sclerosis, psoriasis and allergy.

The at least one cargo moiety which is an antigen (e.g. cancer antigen, pathogen-derived antigen or antigen associated with an immune-mediated disease) may be selected to be recognized by a particular subset of T cells in the subject to be treated, the T cells expressing a TCR known to recognize the chosen antigen. In particular, the antigen may be selected on the basis that it is recognized by T cells used in adoptive cell therapy in the subject to be treated.

For instance, in adoptive cell therapy, T cells may be obtained from the subject, and T cells which recognize an antigen of interest isolated. The isolated T cells may then be expanded and/or otherwise treated to stimulate their effector functionality, and then re-infused into the subject to be treated. In this context, the antigen recognized by the re-infused T cells may be used in the tag construct, by being comprised in the tag construct as at least one cargo moiety. A complex of the disclosure may then be administered to the subject, so that the antigen activates the re-infused T cells.

Alternatively, T cells may be obtained from the subject to be treated or a donor, and genetically modified to express a TCR which recognizes a target antigen. The genetically modified T cells may then be expanded and/or otherwise treated to stimulate their effector functionality, and then infused (or re-infused) into the subject to be treated. In this context, the antigen recognized by the genetically modified T cells may be used in the tag construct, by being comprised in the tag construct as said at least one cargo moiety. A complex of the disclosure may then be administered to the subject, so that the antigen activates the infused T cells. Methods in which administration of a complex of the disclosure is combined with adoptive cell therapy are particularly useful in the treatment of cancer, in which case the antigen used in the tag construct, by being comprised in the tag construct as said at least one cargo moiety, is a cancer antigen.

The disclosure thus provides a method of treating or preventing cancer, comprising administering to a subject a binding molecule of the disclosure, a bispecific conjugate of the disclosure, a complex of the disclosure or a pharmaceutical composition of the disclosure.

In a particular embodiment, the disclosure provides a method of treating cancer in a subject, the method comprising:
(i) obtaining T cells from the subject;
(ii) isolating T cells which recognize a target cancer antigen, and optionally expanding the isolated T cells;
(iii) re-infusing the isolated T cells into the subject; and
(iv) administering to the subject a complex of the present disclosure, wherein the tag construct comprises the target cancer antigen. Equivalently, in step (iv) the subject could alternatively be separately administered a bispecific conjugate of the disclosure and a tag construct comprising the target cancer antigen.

In another embodiment, the disclosure provides a method of treating cancer in a subject, the method comprising:
(i) obtaining T cells from the subject or a donor;
(ii) genetically modifying the T cells to express a TCR which recognizes a target cancer antigen, and optionally expanding the T cells before or after genetic modification;
(iii) infusing the genetically modified T cells into the subject; and
(iv) administering to the subject a complex of the present disclosure, wherein the tag construct comprises the target cancer antigen. Equivalently, in step (iv) the subject could alternatively be separately administered a conjugate of the disclosure and a tag construct comprising the target cancer antigen.

The disclosure similarly provides the use of a binding molecule of the disclosure, a bispecific conjugate of the disclosure or a complex of the disclosure in the manufacture of a medicament for the treatment of prevention of cancer.

Similarly, the disclosure provides a method of treating or preventing an infection, comprising administering to a subject a binding molecule of the disclosure, a bispecific conjugate of the disclosure, a complex of the disclosure or a pharmaceutical composition of the disclosure.

The disclosure also provides the use of a binding molecule of the disclosure, a bispecific conjugate of the disclosure or a complex of the disclosure in the manufacture of a medicament for the treatment of prevention of an infection.

Also, the disclosure provides a method of treating or preventing an autoimmune disease, comprising administering to a subject a binding molecule of the disclosure, a bispecific conjugate of the disclosure, a complex of the disclosure or a pharmaceutical composition of the disclosure.

The disclosure also provides the use of a binding molecule of the disclosure, a bispecific conjugate of the disclosure or a complex of the disclosure in the manufacture of a medicament for the treatment of prevention of an autoimmune disease.

Throughout the above embodiments, reference to the use of a complex of the disclosure includes the combined use of a bispecific conjugate of the disclosure and a tag construct, which are separately or sequentially administered.

In an alternative embodiment, the complex of the disclosure may be used in gene therapy. In this embodiment, a gene therapy vector or delivery system encoding both a bispecific conjugate of the disclosure and a corresponding tag construct may be administered to the subject. Upon take up by cells of the subject, the conjugate and tag construct are expressed and secreted, and form a complex in vivo.

As noted above, the binding molecule of the disclosure, bispecific conjugate of the disclosure or complex of the disclosure may be used as a monotherapy, or in conjunction with other therapeutic agents. Thus, in the treatment of cancer the other therapeutic agent may be an anti-cancer agent, such as a chemotherapeutic agent, numerous classes of which are known in the art, or an immunological agent, including for example, interferons, immune checkpoint inhibitors (e.g. anti-PD-1,-PD-L1 or -CTLA4 antibodies) and other immune-enhancing agents (e.g. anti-OX40 agonistic antibodies). Other therapeutic agents may be beneficial in the treatment of cancer or an infection, e.g. anti-proliferative or anti-inflammatory cytokines, and anti-proliferative, immunomodulatory or factors influencing blood clotting, or inhibitors of angiogenesis. For treatment of an infection, the other (or second) therapeutic agent may be an anti-microbial agent, e.g. an antibiotic, anti-fungal or anti-viral agent.

The binding molecule, bispecific conjugate, complex, or pharmaceutical composition comprising the binding molecule, bispecific conjugate or complex (or the conjugate and tag construct components thereof) may be administered via one or more routes of administration using one or more of a variety of methods known in the art. Similarly, the conjugate and tag construct may be individually administered by these same methods. As will be appreciated by the skilled artisan, the route and/or mode of administration will vary depending upon the desired results. Preferred routes of administration include intravenous, intramuscular, intradermal, intraperitoneal, subcutaneous, spinal or other parenteral routes of administration, for example by injection or infusion, e.g. directly to the site of a tumor.

The phrase “parenteral administration” as used herein means modes of administration other than enteral and topical administration, usually by injection. Alternatively, a non-parenteral route may be used, such as a topical, epidermal or mucosal route of administration. Local administration is preferred, including peritumoral, juxtatumoral, intratumoral, intralesional, perilesional, intra cavity infusion, intravesicle administration, and inhalation. However, the binding molecule, bispecific conjugate, complex or composition may also be administered systemically.

In embodiments where the bispecific conjugate and tag construct are administered individually, i.e. they are not first pre-mixed to form the complex, the conjugate and tag construct must be administered via the same route. Preferably, they are both administered locally, e.g. intradermally, at the same (or substantially the same) site, such that the two components mix, and thus combine to form the complex, rapidly after administration. In these embodiments, the two components must be administered to the subject either simultaneously or rapidly one after the other, avoiding delay between administration of the first component and administration of the second component. This ensures the second component is administered, and complex formation is enabled, before the first component degrades or becomes excessively disseminated from the administration site.

A suitable dosage of a specific binding molecule, bispecific conjugate or complex of the disclosure may be determined by a skilled medical practitioner. Actual dosage levels of the active ingredients in the pharmaceutical compositions and products of the present disclosure may be varied so as to obtain an amount of the active ingredient which is effective to achieve the desired therapeutic response for a particular subject, i.e. patient, without being toxic to the patient. The selected dosage level will depend upon a variety of pharmacokinetic factors including the activity of the particular complex/conjugate employed, the route of administration, the time of administration, the rate of excretion of the complex, the duration of the treatment, other drugs, compounds and/or materials used in combination with the particular compositions employed, the age, sex, weight, condition, general health and prior medical history of the patient being treated, and like factors well known in the medical arts.

A suitable dose of a binding molecule, bispecific conjugate or complex of the disclosure may be, for example, in the range of from about 0.1 μg/kg to about 100 mg/kg body weight of the patient to be treated. For example, a suitable dosage may be from about 0.1 μg/kg to about 10 mg/kg body weight per day or from about 10 μg/kg to about 5 mg/kg body weight per day.

Dosage regimens may be adjusted to provide the optimum desired response (e.g. a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The binding molecule, bispecific conjugate or complex (or combination of the conjugate and tag construct) may be administered in a single dose or in multiple doses. The multiple doses may be administered via the same or different routes and to the same or different locations. Alternatively, complexes can be administered as a sustained release formulation, in which case less frequent administration is required. Dosage and frequency may vary depending on the half-life of the administered species in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms of disease. In an exemplary dosage regime, the complex (or combination of the conjugate and tag construct) is administered to the subject once a week, once a fortnight or once every three weeks, in a cycle repeated from 2 to 10 times.

Combined administration of two or more agents may be achieved in a number of different ways. In one embodiment, the complex and the other agent may be administered together in a single composition. In another embodiment, the complex and the other agent may be administered in separate compositions as part of a combined therapy. For example, the complex may be administered before, after or concurrently with the other agent. The complex of the disclosure may be administered in combination with or sequentially to tumor targeting antibodies, target therapy, pathway inhibitors or other immunomodulatory antibodies targeting e.g. PD-1, PD-L1, CD137, GITR, OX40, CTLA-4, CD27, HVEM, LTβR, and LAG3. Further the complex may also be combined with local radiation. Similarly, such additional therapies may be co-administered when the conjugate and tag construct are individually administered to the subject.

The disclosure also provides an in vitro or ex vivo method of activating a T cell expressing a TCR which recognizes an antigen, said method comprising contacting an antigen-presenting cell with:
i) a bispecific conjugate of the disclosure and a tag construct as defined above, wherein said tag construct comprises the antigen recognized by said TCR; or
ii) a complex of the disclosure, wherein the tag construct of said complex comprises the antigen recognized by said TCR.

Thus, the complex or bispecific conjugate of the disclosure may be used to activate APCs in vitro or ex vivo, as well as in vivo. The complex and bispecific conjugate (in combination with a tag peptide comprised in a tag construct) thus have both medical and non-medical uses, and all such uses are encompassed herein. For example, isolated or cultured APCs may be contacted with the complex, e.g. in a laboratory setting, for example for research, development or testing purposes. This may be achieved by pre-mixing the conjugate and tag construct, to form the complex, and then applying the complex to the APCs. Alternatively, the conjugate and tag construct can be individually applied to the APCs, such that the complex forms within the APC culture.

The complex may be used to activate a T cell which expresses a TCR which recognizes the antigen comprised in the tag construct. Specifically, the TCR recognizes the antigen when presented by an APC (i.e. in the context of an MHC). Thus, an APC activated by the complex, or for activation by the complex, may be contacted with the T cell. Thus for example, APCs may be cultured or incubated in the presence of the complex (or constituent parts thereof), following which the APCs may be contacted with the T cells, e.g. co-cultured, or further incubated in the presence of the T cells. Alternatively, the complex (or constituent parts thereof), APCs and T cells may be incubated or co-cultured together. Thus the antigen is delivered to the APCs and presented to the T cells, resulting in their activation.

The invention is further illustrated by the following non-limiting figures and examples:

### BRIEF DESCRIPTION OF THE FIGURES

FIG. 1 shows a sequence alignment illustrating the design and construction of a humanized version of the mouse scFv IBIIICI as described in Example 1. From top to bottom: VH and VL sequences from wild type murine scFv IBIIICI (“VH_Murine”: SEQ ID NO:55; “VL_Murine”: SEQ ID NO:56); the selected human germline framework sequences; the sequences resulting from grafting of the mouse scFv CDR:s into the human germline frameworks (VH: SEQ ID NO:6) and VL (SEQ ID NO:10) respectively.

FIG. 2 shows an SDS-PAGE gel of scFv expressed and purified as described in Example 2. M: molecular weight ladder. Lane 1: IBIIICI mouse scFv (SEQ ID NO:25). Lane 2: IBIIICI CDR graft scFv (SEQ ID NO:54)

FIG. 3 shows sensorgrams obtained from affinity measurements by SPR of the interaction of immobilized IBIIICI mouse scFv (left-hand side; A, C, E; SEQ ID NO: 25) or IBIIICI CDR graft scFv (right-hand side; B, D, F; SEQ ID NO:54) against a panel of 14 peptides (A-B: UU0070-UU0074, SEQ ID NO:27-31; C-D: UU0075-UU0078, SEQ ID NO:32-35; and E-F: UU0024, UU0080-UU0083, SEQ ID NO:26, 36-39) as described in Example 3.

FIG. 4 shows a sequence alignment of the VH sequences of mouse IBIIICI (SEQ ID NO:55), the original IBIIICI CDR graft VH (SEQ ID NO:6) and five CDR graft variant VH:s with mutations in a putative deamidation site, denoted IBIIICI CDR graft GG (SEQ ID NO:2), IBIIICI CDR graft DG (SEQ ID NO:4), IBIIICI CDR graft SG (SEQ ID NO:1), IBIIICI CDR graft QG (SEQ ID NO:3) and IBIIICI CDR graft NA (SEQ ID NO:5). Boundaries for CDRs and residue numbering are as defined by the IMGT nomenclature (Lefranc et al., 2003, Dev Comp Immunol 27 (1):55-77).

FIG. 5 shows an SDS-PAGE gel of the indicated scFv variants, expressed and purified as described in Example 4.

FIG. 6 shows sensorgrams obtained from the six indicated IBIIICI CDR graft scFv variants when analyzed for the interaction to five peptides as described in Example 5. The analyzed scFv variants are denoted WT (SEQ ID NO:54); SG (SEQ ID NO:49); GG (SEQ ID NO:50); QG (SEQ ID NO:51); DG (SEQ ID NO:52) and NA (SEQ ID NO:53). The analyzed peptides were A) UU0024 (SEQ ID NO:26); B) UU0084 (SEQ ID NO:40); C) UU0085 (SEQ ID NO:41); D) UU0086 (SEQ ID NO:42); and E) UU0087 (SEQ ID NO:43).

FIG. 7 shows SPR sensorgrams obtained from the six indicated IBIIICI CDR graft scFv variants when analyzed for the interaction to four peptides as described in Example 5. The analyzed scFv variants are denoted WT (SEQ ID NO: 54); SG (SEQ ID NO:49); GG (SEQ ID NO:50); QG (SEQ ID NO:51); DG (SEQ ID NO:52) and NA (SEQ ID NO:53). The analyzed peptides were A) UU0090 (SEQ ID NO:44); B) UU0091 (SEQ ID NO:45); C) UU0092 (SEQ ID NO:46); D) negative control p003 (SEQ ID NO:48).

FIG. 8 shows the results of the western blot experiment on the indicated bispecific conjugate constructs SP031 and SP007, as described in Example 6. Analysis was performed on supernatants from 2.5 ml expression cultures taken 5 days post transfection.

FIG. 9 shows the results of the biolayer interferometry experiment on the indicated bispecific conjugate constructs SP031 and SP007, as described in Example 6.

FIG. 10 is a survival graph showing the probability of survival in the indicated three groups of mice in the experiment described in Example 7. HD: high dose. LD: low dose.

### EXAMPLES

The following examples disclose the development of novel binding molecules, IBIIICI CDR graft scFv:s, created by grafting of the complementarity determining regions (CDR:s) from the known mouse scFv IBIIICI into human germline variable heavy (VH) and variable light (VL) frameworks and additional point mutations. The amino acid sequences of the humanized VH and VL domains designed as described herein are listed in the sequence listing as SEQ ID NO:1-6 (VH sequences) and SEQ ID NO:10 (VL sequence), and the complete scFv sequences are listed as SEQ ID NO: 49-54. The examples further describe the expression and characterization scFv polypeptides and variants thereof, as well as bispecific conjugates and complexes comprising them, and demonstrate their surprisingly superior properties in terms of expression and function in both bacterial and mammalian expression settings.

### Example 1

### Design and Construction of a Humanized Version of IBIIICI Mouse scFv

Mangsbo et al (Mol Immunol 93:115-124, 2018) describe the creation of mouse scFv IBIIICI, which is further studied in a bispecific conjugate context in WO2020/104690 and WO2021/239968. The complete amino acid sequence of mouse scFv IBIIICI is listed as SEQ ID NO:25 herein (SEQ ID NO:48 in WO2020/104690). In this disclosure, humanization of this ancestral mouse scFv was performed via CDR grafting into human germline variable heavy and variable light (VH/VL) frameworks as described below.

### Materials and Methods

Design of the IBIIICI CDR Graft scFv

Human germline VH sequence IGHV1-46, which has a sequence homology of 59.4% to IBIIICI mouse VH (residues 1-104; IMGT/DomainGapAlign), was chosen as the VH framework. For choice of VL, sequence homology was considered, but VH/VL pairings with favorable biophysical properties were also taken into account (Tiller et al, 2013, mAbs 5(3):445-470). Taken together, this resulted in the selection of the human germline gene IGKV1-39 for the VL graft. Based on the IMGT/DomainGapAlign domain search, IGHJ4 and IKVJ2 were chosen as joining fragments for VH and VL, respectively.

Construction of the IBIIICI CDR Graft scFv

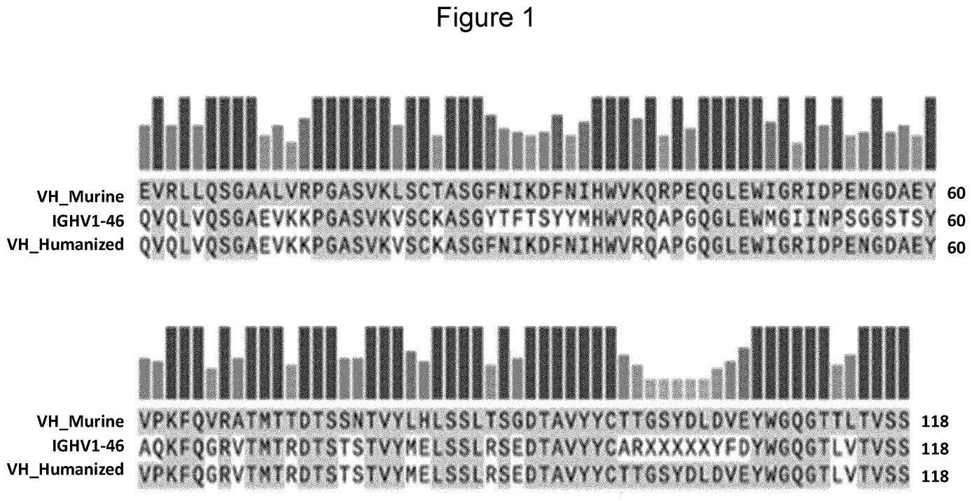

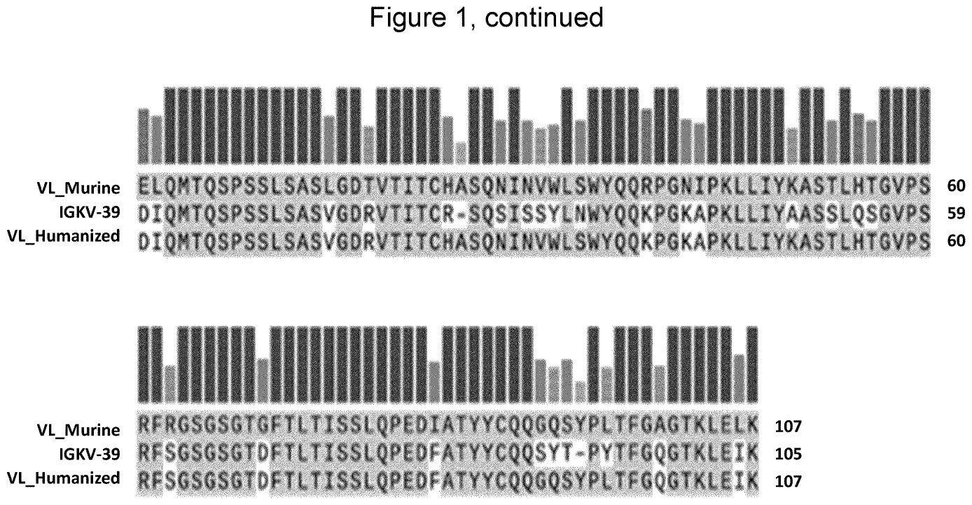

The IBIIICI CDR graft was created by grafting the six antigen binding loops of the mouse sequence onto the human IGHV1-46/IGKV1-39 scaffold (FIG. 1). A gene encoding the VH fused to the VL via a glycine-serine linker ((G₄S)₄; SEQ ID NO:14) was designed. Nucleotides encoding two additional amino acids (R and T, both part of the light chain constant domain) were added at the end of the VL gene in order to include a BsiWI restriction site. Synthesis and sub-cloning of the scFv gene were performed (GenScript, Piscataway, USA) without codon optimization. For the framework regions, the nucleotide sequences found in the human germline genes was used. In the CDRs, the codons found in the original mouse antibody were used. After synthesis, the scFv gene was cloned into an in-house vector using restriction enzymes SfiI and BsiWII, providing the scFv with a triple-FLAG tag and a hexahistidine tag at the C-terminus when expressed.

### Results

Design of the IBIIICI CDR Graft scFv

The degree of humanness introduced during the humanization procedure of the IBIIICI mouse scFv was calculated using the T20 score analyzer (Gao et al., 2013, BMC Biotechnology 13:55). Briefly, the T20 score is scaled from 0 to 100, where a higher score represents a more human-like antibody. The T20 scores obtained for the original IBIIICI mouse scFv and of the IBIIICI CDR graft scFv are presented in Table 1.

TABLE 1

| | IBIIICI mouse scFv | IBIIICI CDR scFv graft |
|---|---|---|
| Region | T20 score | T20 score |
| VH, Framework + CDRs | 58.7 | 77.0 |
| VH, Framework only | 66.0 | 90.4 |
| VL, Framework + CDRs | 76.1 | 86.0 |
| VL, Framework only | 84.6 | 99.0 |

The T20 scores for the VH and VL sequences of the humanized scFv IBIIICI CDR graft all indicated clear increases in humanness compared to the T20 scores for their respective mouse counterparts. T20 scores for the VH framework and VL framework for IBIIICI CDR graft were both >90, surpassing the criterium of a T20 score above 85 to be considered “human-like”. Based on the comparison of the T20 scores between the original IBIIICI mouse scFv and the resulting IBIIICI CDR graft scFv, the humanization procedure was determined to be successful.

Construction of the IBIIICI CDR Graft scFv

Following gene synthesis and cloning, 4 μg of vector encoding the IBIIICI CDR graft scFv was obtained. DNA sequencing verified the correct sequence.

### Example 2

### Small-Scale Expression and Purification of IBIIICI Mouse scFv and IBIIICI CDR Graft scFv

This example describes small-scale expression and purification of both the known IBIIICI mouse scFv (SEQ ID NO:25) and the newly designed and constructed humanized version, IBIIICI CDR graft scFv (SEQ ID NO:54). Expression and purification were performed and the results compared in order to determine whether the humanization process had affected the developability of the scFv construct.

### Materials and Methods

Vectors encoding the scFv variants described in Example 1 were transformed into Top10 E. coli and the intended sequence confirmed by sequencing.

50 ml cultures comprising the respective scFv vectors were grown overnight, the cultures centrifuged and the pellets resuspended in B-Per Protein Extraction Buffer (Pierce). Following another round of centrifugation, a Ni-NTA Sepharose resin slurry (GE Healthcare) was added to the cleared supernatants. After incubation, the mixtures were transferred to empty gravity flow columns. After washing, the expressed scFv proteins were eluted with buffer containing imidazole, and the samples were buffer exchanged and concentrated to approximately 0.5 ml in PBS. Protein concentration was determined with an Implen Nanophotometer, and purity and integrity were verified by SDS-PAGE.

### Results

The IBIIICI CDR graft scFv showed a satisfying purity when evaluated by

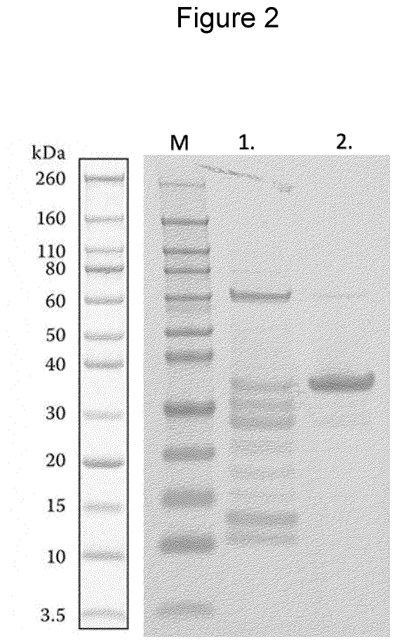

SDS-PAGE, as shown by the presence of one main band correlating to the expected molecular weight of an scFv (appr 30 kDa; FIG. 2). In addition, the measured protein concentration of the IBIIICI CDR graft scFv sample was relatively high (1.2 mg/ml). The IBIIICI mouse scFv, on the other hand, displayed a low sample purity with multiple protein bands derived from E. coli. Furthermore, the protein band corresponding to an scFv (approximately 30 kDa) for the IBIIICI mouse scFv was quite weak in comparison to the other bands on the gel, indicating a much lower concentration of this product than the measured value (0.2 mg/ml).

### Conclusion

The small-scale expression experiment indicated that IBIIICI CDR graft scFv has a superior expression profile compared to its mouse ancestor.

### Example 3

### Measurement of Binding Properties of Mouse IBIIICI scFv and IBIIICI CDR Graft scFv Using Surface Plasmon Resonance

The binding kinetics of the novel IBIIICI CDR graft scFv was compared to the binding kinetics of the IBIIICI mouse scFv towards a panel of peptides using surface plasmon resonance (SPR).

### Materials and Methods

Affinity measurements of the IBIIICI mouse scFv and IBIIICI CDR graft scFv variants obtained as described in Examples 1-2 were performed by SPR using a Biacore T200 instrument (GE Healthcare) and single cycle kinetics. Anti-FLAG M2 antibody (Sigma-Aldrich) was immobilized onto a Series S CM5 chip by primary amine coupling using NHS-EDC chemistry according to the manufacturer's instruction, allowing capture of the respective scFv through the FLAG tags provided by the expression vector. A 5-fold dilution series comprising of five concentrations (0.16 nM to 100 nM) of different peptides (Table 2) was sequentially injected over the flow cells, allowing binding to the captured scFv. Following a dissociation phase, regeneration of the surface was accomplished under acidic conditions using 10 mM glycine-HCl at pH 2.1. By subtracting the response curve of a surface having an anti-FLAG antibody immobilized thereto (the reference surface), response unit sensorgrams for all peptides were obtained. Data was analyzed using BIAeval v.3.1 (GE Healthcare).

TABLE 2
Panel of peptides used in Examples
3 and 5, with tag moiety in bold

| | SEQ | |
|---|---|---|
| | ID | |
| Designation | NO: | Amino acid sequence |
| UU0024 | 26 | FIGITELKKLESKINKVFK-Biotin |
| UU0070 | 27 | FIGITELKKLESKINK |
| UU0071 | 28 | FIGITELKKLESKIN |
| UU0072 | 29 | FIGITELKKLESKI |
| UU0073 | 30 | FIGITELKKLESK |
| UU0074 | 31 | FIGITELKKLES |
| UU0075 | 32 | LEQLESIINFEKLLAAAAA |
| | | KFIGITELKKLES |
| UU0076 | 33 | FIGITELKKLESLEQLESI |
| | | INFEKLLAAAAAK |
| UU0077 | 34 | Biotin-(PEG3)FIGITELKKLES |
| UU0078 | 35 | FIGITELKKLES(PEG3)K-Biotin |
| UU0080 | 36 | FIGITELHHLESK-Biotin |
| UU0081 | 37 | FIGISELKKLESK-Biotin |
| UU0082 | 38 | FIGITELHKLESK-Biotin |
| UU0083 | 39 | FIGITELKHLESK-Biotin |
| UU0084 | 40 | FIGITELKKLE |
| UU0085 | 41 | FIGITELKKL |
| UU0086 | 42 | FIGITELKK |
| UU0087 | 43 | FIGITELK |
| UU0090 | 44 | FIGITELLEQLESIINFEKLAAAAAK |
| UU0091 | 45 | FIGITELKLEQLESIINFEKLAAAAAK |
| UU0092 | 46 | FIGITELHLEQLESIINFEKLAAAAAK |
| UU0088 | 47 | ANSKFIGITELK |
| p003 | 48 | Biotin-IDIKNDLYEKTLNDYKAIA |
| | | NKLSQV |

### Results

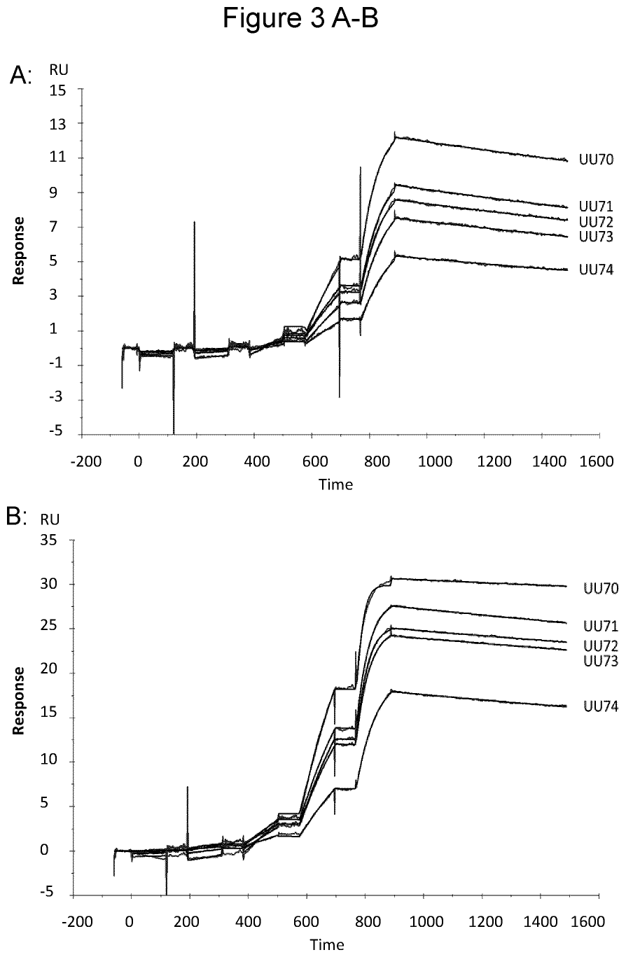

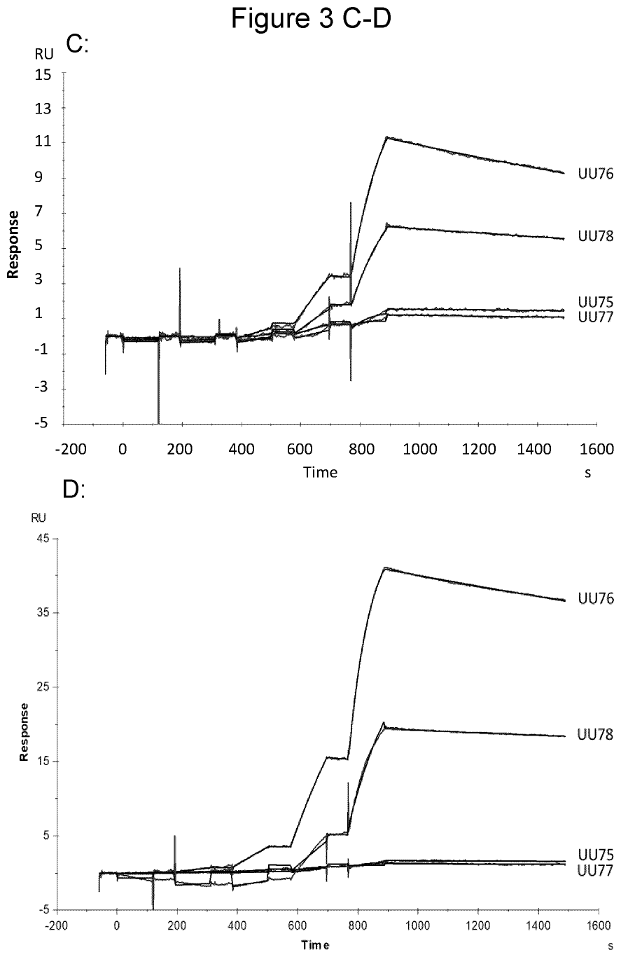

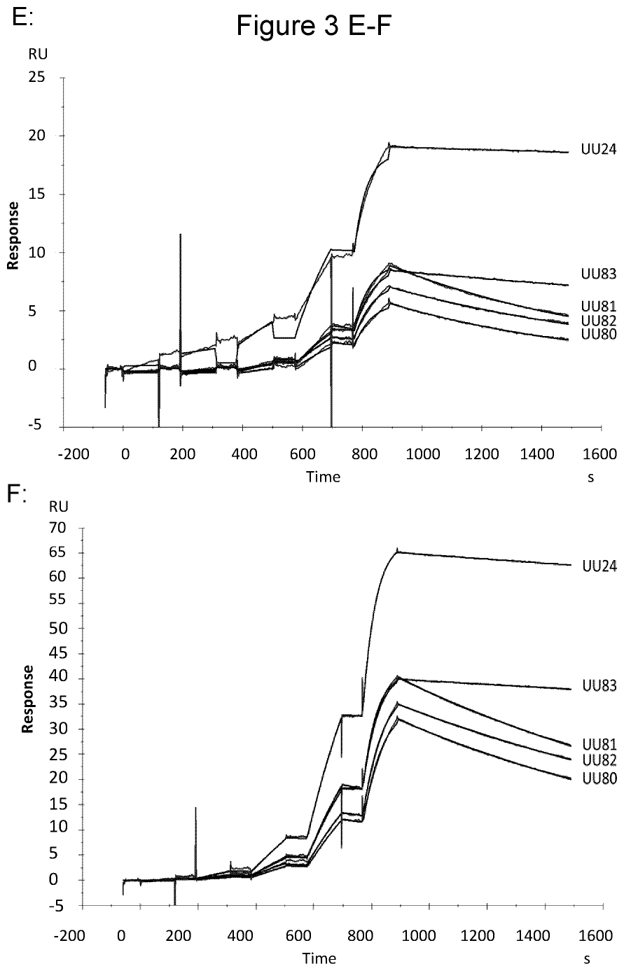

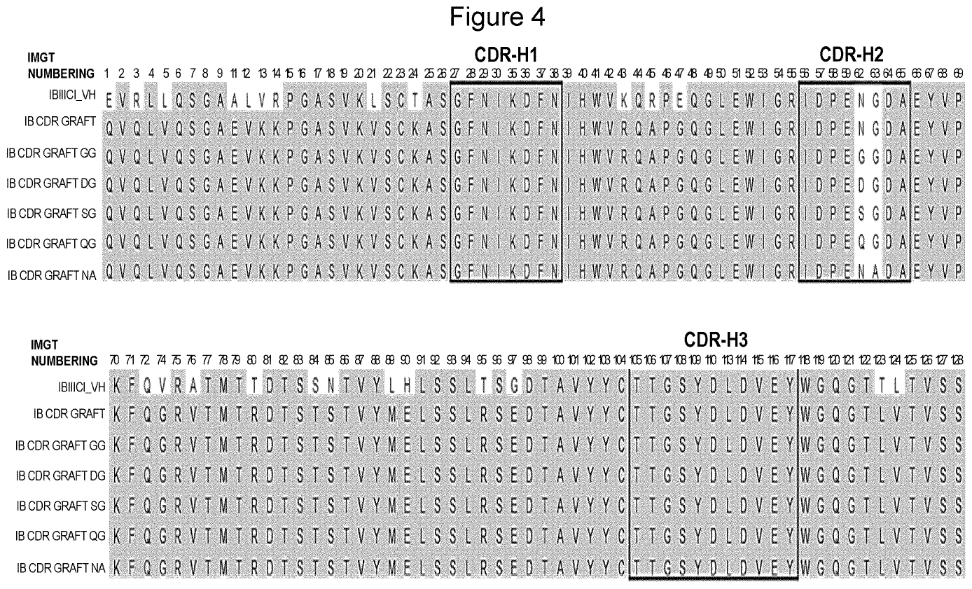

The results are presented in FIG. 3. Overall, the binding properties of the IBIIICI mouse scFv (FIGS. 3A, 3C and 3E) and the humanized IBIIICI CDR graft scFv (FIGS. 3B, 3D and 3F) were very similar, with low nanomolar binding affinity to most target peptides in the tested panel, indicating a successful humanization.

### Example 4

### Generation of Five IBIIICI CDR Graft scFv Variants by Point Mutations

A putative deamidation site is present in the CDR-H2, at positions 62-63 of the IBIIICI VH sequence. With the goal of mutating this site without negatively affecting the binding properties or other important biophysical parameters of the humanized IBIIICI scFv, five point mutation variants were created.

### Materials and Methods

Design and Construction of Five IBIIICI CDR Graft scFv Variants

Five different versions of the CDR graft VH sequence with a mutated deamidation site were designed. The diversity found in natural antibody repertoires at these positions was taken into consideration (Kirik et al. 2017, Front Immunol 8:1433; Persson et al. 2018, Front Immunol 9:1391). Thereafter, conservative replacements were made, i.e. amino acid replacements to different amino acids with similar biochemical properties. Alignment of the VH sequences of the original mouse scFv, the humanized scFv described in Examples 1-3 and the five point mutated variants is presented in FIG. 3. The amino acid sequences of the respective VH domains of the five point mutated variants and of the original humanized variant are represented by SEQ ID NO:1-6 in the sequence listing. The complete scFv sequences of the five point mutated variants were denoted IB CDR graft scFv SG (SEQ ID NO:49), IB CDR graft scFv GG (SEQ ID NO:50), IB CDR graft scFv QG (SEQ ID NO:51), IB CDR graft scFv DG (SEQ ID NO:52) and IB CDR graft scFv NA (SEQ ID NO:53). The “original” humanized IBIIICI CDR graft scFv (SEQ ID NO:54) was denoted “IB CDR graft scFv NG” or “IB CDR graft scFv WT” and comprises the original amino acids NG in the CDR-H2 at positions 62-63 of the VH sequence (FIG. 3).

The genes encoding the scFv variants were constructed by introducing point mutations into the IBIIICI CDR graft scFv vector designed as described in Example 2. The vector provided the scFv clones with a triple-FLAG tag and a hexahistidine tag at the C-terminus.

### Small-Scale Expression and Purification

The vectors encoding the designed scFv constructs were transformed into Top10 E. coli and the intended sequences were confirmed by sequencing.

50 ml cultures of the six different humanized scFv variants and the original IBIIICI mouse scFv were grown overnight, the cultures centrifuged and the pellets resuspended in B-Per Protein Extraction Buffer (Pierce). Following another round of centrifugation, a Ni-NTA sepharose resin slurry (GE Healthcare) was added to the cleared supernatants. After incubation, the mixtures were transferred to empty gravity flow columns. After washing, the polypeptides were eluted with buffer containing imidazole, and the samples were buffer exchanged and concentrated to approximately 0.4 ml in PBS. Protein concentration was determined with an Implen Nanophotometer and purity and integrity verified by SDS-PAGE.

### Results

Bacterial expression levels between the five different point-mutated IBIIICI CDR graft scFv variants and the IBIIICI CDR graft WT scFv (“variant NG”) were relatively high and very similar. This is in contrast to the IBIIICI mouse scFv, which produced relatively poorly (as also shown previously in Example 2).

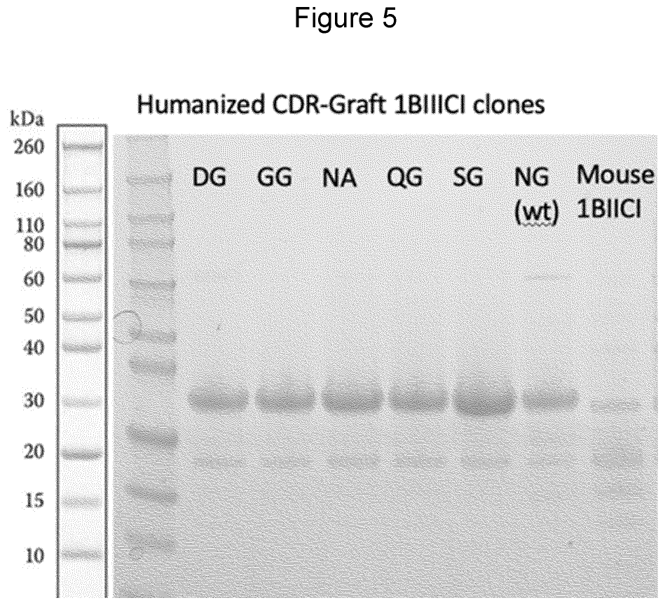

The SDS-PAGE gel is shown in FIG. 5. The IBIIICI mouse scFv displayed a low sample purity with multiple E. coli derived protein bands. Furthermore, the protein band corresponding to the size of an scFv (approximately 30 kDa) in the IBIIICI mouse scFv lane was quite weak in comparison to the other bands on the gel, indicating a much lower concentration of this clone. All point mutant variants exhibited a satisfying purity, as evidenced by one main band corresponding to the expected molecular weight of an scFv (appr 30 kDa). In addition, the measured protein concentrations of these samples were relatively high (2.3-3.3 mg/ml). The expression and purification of the variants was unaffected when compared to the IBIIICI CDR graft WT scFv (“variant NG”), and therefore, in terms of expression and purification, the creation of point mutated variants was considered successful.

### Example 5

### Biophysical Characterization of IBIIICI CDR Graft scFv Variants

The five IBIIICI CDR graft scFv point-mutated variants created as described in Example 4 and the IBIIICI CDR graft WT scFv described in Examples 1-3 were characterized using three different biophysical characterization methods: SPR, nanoDSF and size exclusion chromatography (SEC).

### Materials and Methods

### Surface Plasmon Resonance

Affinity measurements of IBIIICI CDR graft WT scFv and IBIIICI CDR graft scFv point-mutated variants were performed by SPR using a Biacore T200 instrument (GE Healthcare) and single cycle kinetics. The anti-FLAG antibody M2 (Sigma-Aldrich) was immobilized onto a CM5 S chip by primary amine coupling using NHS-EDC chemistry, allowing capture of the respective scFv through their FLAG tags. A five-fold dilution series comprised of five concentrations (0.16 nM to 100 nM) of a panel of tested peptides (Table 2) was sequentially injected over the flow cells, allowing binding to the respective captured scFv. Following a dissociation phase, regeneration of the surface was performed under acidic conditions using 10 mM glycine-HCl at pH 2.1. By subtracting the response curve of a reference surface having an immobilized anti-FLAG antibody, response unit sensorgrams for all peptides were obtained. Data was analyzed using BIAeval v.3.1 (GE Healthcare).

Melting Temperature (Tm) Determination by nanoDSF

In order to evaluate the stability of the purified scFv variants, the melting temperatures were determined by nanoDSF using a Prometheus NT.48 instrument (NanoTemper Inc). The nanoDSF technology measures the intrinsic fluorescence of a protein while it is being subjected to thermal denaturation, thereby characterizing the unfolding of the protein under native conditions.

The six purified IBIIICI CDR graft scFv variants were diluted to 1.0 mg/ml in PBS and loaded into a high sensitivity capillary (NanoTemper, #PR-C006) through capillary force. A melting temperature ramp was set to between 20° C. and 95° C., with heating at 1° C./min. Tryptophan emission was measured at 330 nm and 350 nm, and the calculated ratio was plotted against temperature to obtain melting curves for each variant. Tm values were deduced using the software PR ThermoControl (NanoTemper Inc).

### Stability Study and SEC

In order to further assess any differences in stability between the variants, a stability assessment was made. The six purified IBIIICI CDR graft scFv variants were analyzed by SEC HPLC using a Bio SEC-3 3 μm 300 A column (Agilent). Samples were analyzed at two time points following incubation at 37° C.: t=0 h and t=24 h.

### Results

### Surface Plasmon Resonance

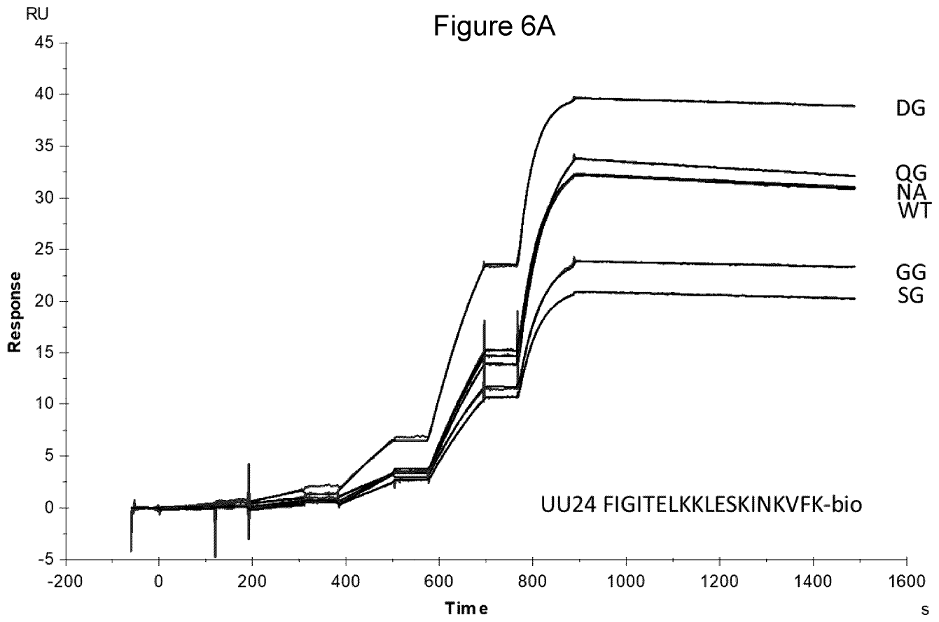

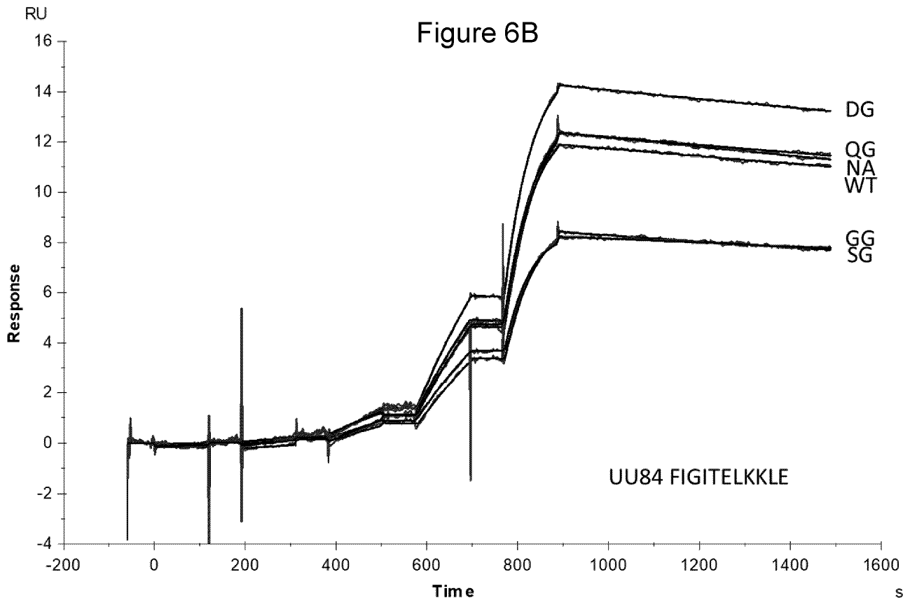

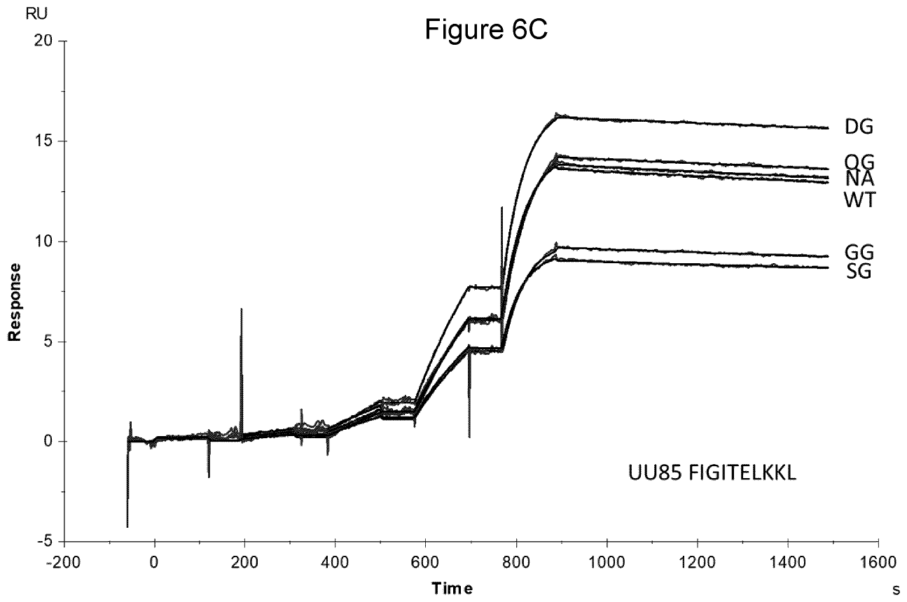

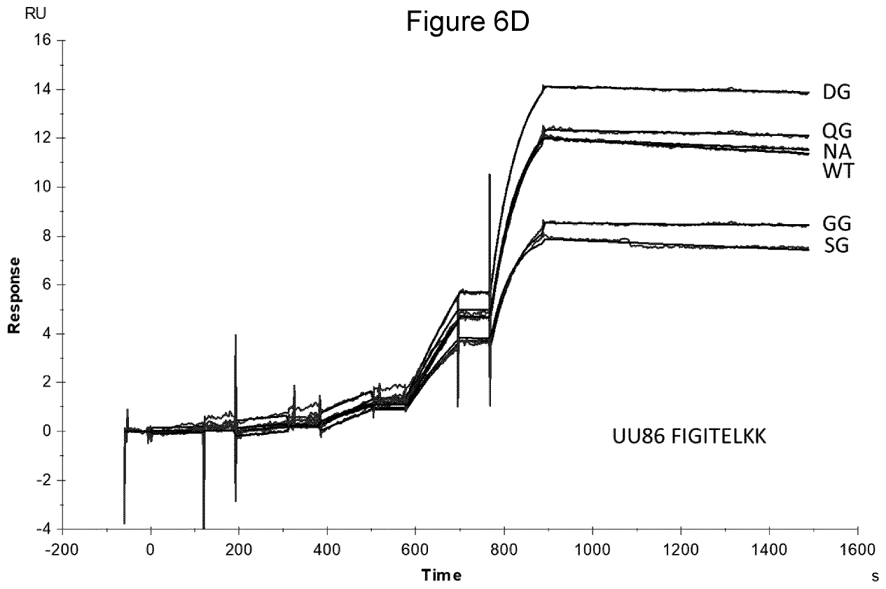

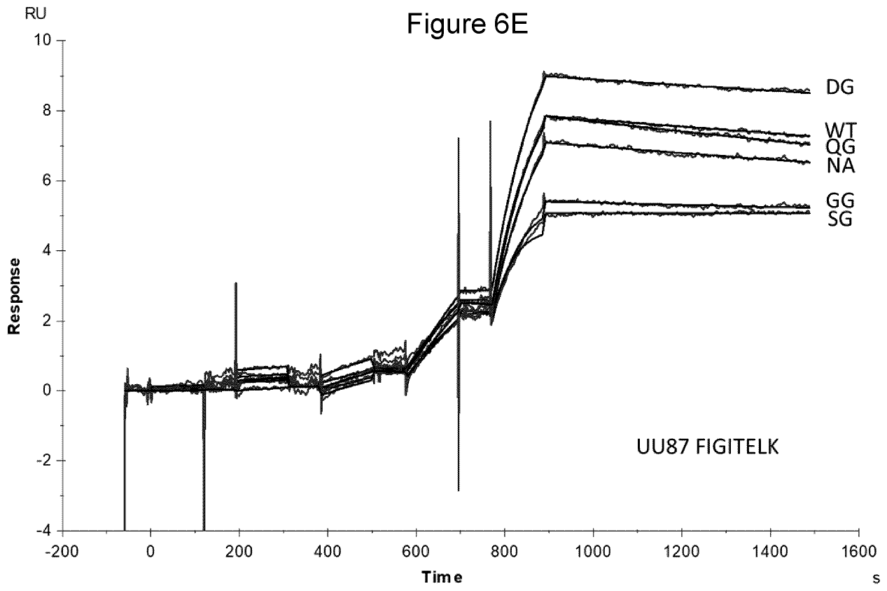

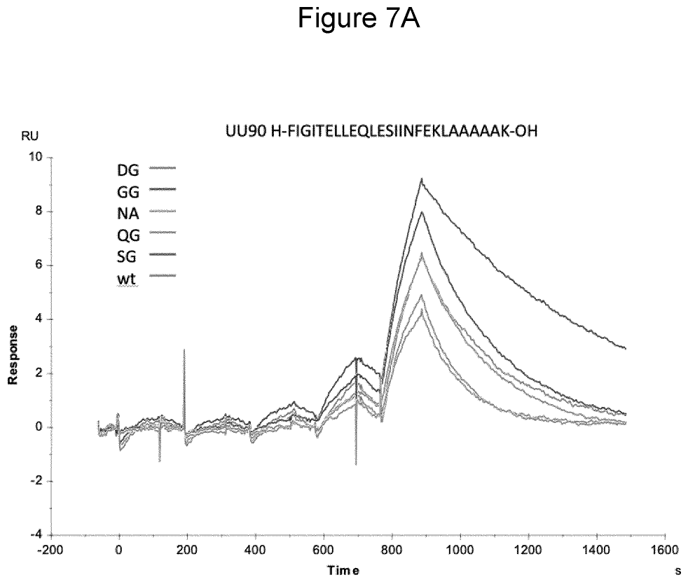

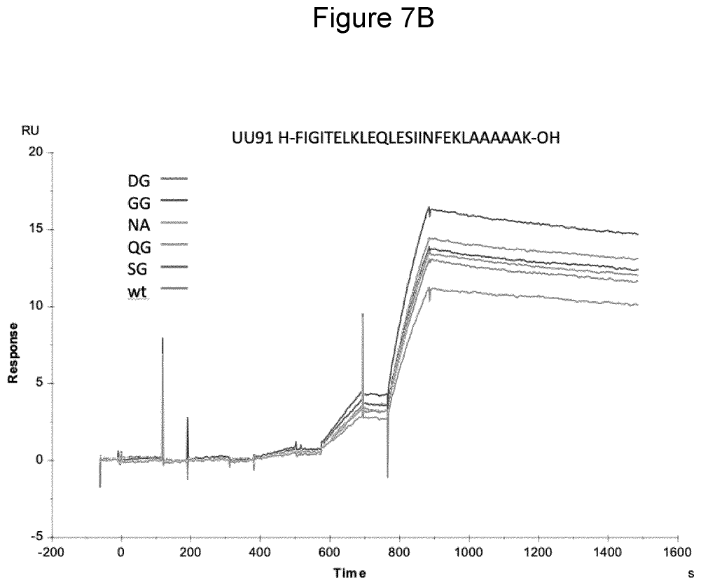

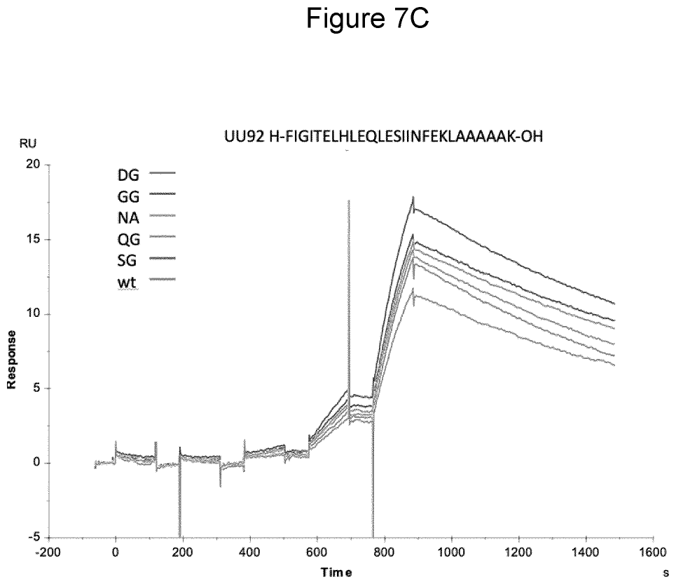

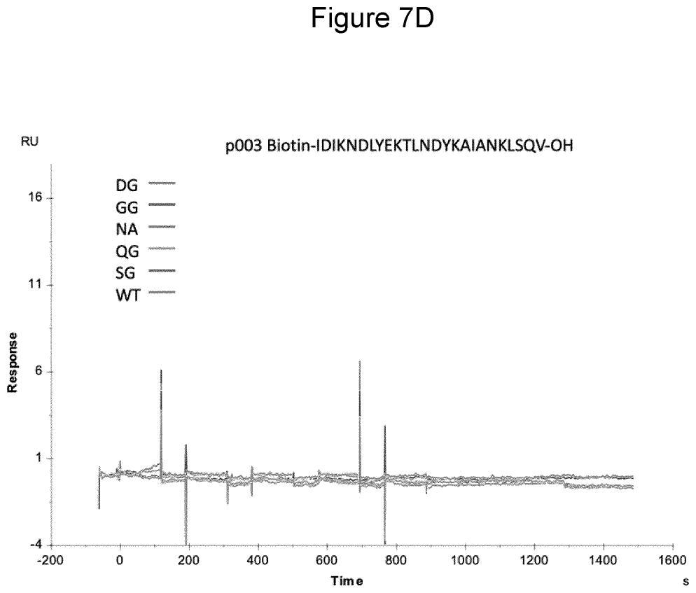

The results are shown in FIGS. 6-7. Overall, the binding properties of the point mutated IBIIICI CDR graft scFv variants towards the panel of peptides were very similar to each other as well as to the IBIIICI CDR graft WT (variant “NG”), indicating that the introduced point mutations in positions 62 and 63 of the VH sequence do not negatively influence binding. FIGS. 6A-E show the binding sensorgrams of the different IBIIICI CDR graft scFv variants towards peptides UU0024, UU0084, UU0085, UU0086 and UU0087 are shown, while FIGS. 7A-C show the corresponding binding to peptides UU0090, UU0091 and UU0092 (see Table 2 for peptide sequences). No variant displayed any binding to the negative control peptide p003 (FIG. 7).

### NanoDSF Measurements

The measured Tm values (inflection point #1) are shown in Table 3, and were between 6° and 65° C. Determined Tm values are useful as a measure of protein stability. The data indicated a somewhat higher stability for variant IBIIICI CDR graft scFv SG (64° C.) compared to the other analyzed scFv variants. In contrast, variant IBIIICI CDR graft scFv NA had the lowest Tm value in the set (61° C.).

TABLE 3
Melting temperature (Tm) values obtained by nanoDSF

| scFv | SEQ ID NO: | Tm (° C.) |
|---|---|---|
| IBIIICI CDR graft scFv variant DG | 52 | 62.4 |
| IBIIICI CDR graft scFv variant GG | 50 | 62.6 |
| IBIIICI CDR graft scFv variant QG | 51 | 62.2 |
| IBIIICI CDR graft scFv variant SG | 49 | 64.3 |
| IBIIICI CDR graft scFv variant NA | 53 | 60.8 |
| IBIIICI CDR graft scFv variant NG (wt) | 54 | 63.4 |

### SEC HPLC Analysis

To investigate the integrity and stability of the CDR graft variants, the six IBIIICI CDR graft scFv variants were analyzed by SEC HPLC before and after incubation at 37° C. for 24 h. No detectable change in monomeric content was visible for any of the IBIIICI CDR graft scFvs after 24 h compared to the start samples.

### Conclusion

The point mutations introduced in CDR-H2 have minimal, if any, effect on the binding of scFv variants to the tested peptides or on the stability of the scFv variants. Each of the five CDR graft variants designed to remove a putative deamidation site shows a very similar binding pattern compared to both the first IBIIICI CDR graft scFv (variant “NG”) and to the original IBIIICI mouse scFv.

### Example 6

### Construction, Expression and Characterization of Bispecific Conjugates of Murine and Humanized IBIIICI scFv:s to a Full-Length Antibody

In this Example, a humanized scFv according to this disclosure is placed in the bispecific conjugate context described in WO2020/104690 and WO2021/239968, i.e. a humanized scFv is designed to form part of a bispecific conjugate construct wherein the peptide binding scFv is conjugated to another binding molecule, e.g. a full-length antibody against CD40. See FIG. 6A of WO2020/104690 for a schematic illustration of the construct design. The anti-CD40 antibody used in this Example is the antibody denoted “A9” disclosed in WO2021/239968. Expression and purification in the conjugate context of both the IBIIICI mouse scFv and two humanized versions, IBIIICI CDR graft scFv NG and IBIIICI CDR graft scFv SG, was performed and the results compared.

### Materials and Methods

Expression of Murine and Humanized scFv-A9 Conjugates

Four bispecific conjugate constructs (Table 4) were expressed in ExpiCHO-S™ cells (Gibco, #A29127) according to manufacturer's instructions, where the 7-day standard expression protocol was used. The bispecific conjugates were expressed in 2.5 ml transfection volume, incubated at 37° C., 5% CO₂, humidified air and 250 rpm. Supernatant was harvested 10 days after transfection for purification.

Conjugates were then transfected and expressed in scaled up 25 ml cultures, incubated at 37° C., 5% CO₂, humidified air and 120 rpm, and harvested 7 days post transfection for affinity purification.

### Western Blot

A total of 20 μl harvested supernatant from day 5 post transfection was mixed with 3× loading buffer (0.1 M Tris-HCl, 45% glycerol, 0.03% bromophenol blue, 0.3% SDS) for non-reducing conditions. The samples were run on a 4-20% Criterion™ TGX Stain-Free™ protein gel (Bio-Rad Laboratories, #5678095) according to the manufacturer's protocol. Gels were transferred to PVDF membranes (BioRad Transfer-Blot Turbo transfer system) for western blot analysis. The membrane was incubated for 1 h in 5% TBST milk buffer for 1 h, followed by 1 h incubation with goat-anti human-HRP antibody (ThermoFisher). Membranes were washed 3×5 min in TBST prior to incubation with chemiluminescent substrate (Merck) and analysis of the blots in a Chemidoc XRS+system (BioRad).

### Biolayer Interferometry

Titers in the supernatant from 5 days post transfection were determined by biolayer interferometry measurements in an Octet® RED96e system with Dip and Read™ Protein A biosensors (Fortebio Biologics by Molecular Devices, #10819702) according to the manufacturer's instructions. Supernatants were diluted 1:1 with an end concentration of the equilibration buffer at 20 mM citric acid pH 4.0, 0.1% BSA (w/v), 0.1% Tween-20, 0.5 M NaCl to a final volume of 200 μl in 96 well black plates. A standard curve was prepared from an IgG (IgG1 Protein A Standard, Sartorius #18-1118) in a concentration span from 1 to 700 μg/ml.

TABLE 4
List of expressed bispecific constructs

| | Anti-CD40 Ab HC linked | Anti-CD40 | |
|---|---|---|---|
| | to peptide-binding scFv | Ab LC | |
| Designation | (SEQ ID NO) | (SEQ ID NO) | scFv type |
| SP031 | 57 | 58 | mouse |
| SP007 | 59 | 58 | humanized |
| SP019 | 60 | 58 | humanized |
| SP027 | 60 | 61 | humanized |

Purification of Murine and Humanized scFv-A9 Conjugates

The expressed conjugates were purified by Protein A affinity chromatography on an ÄktaSTART system using mAbSelect SuRe columns (GE Healthcare, #11003493). A 20 mM sodium phosphate, 0.15 M sodium chloride (pH 7.3) buffer was used as binding and wash buffer, 0.1 M glycine (pH 2.5) as elution buffer and 1M Tris-HCl (pH 8.5) as neutralization buffer.

### Purity Measurements of Conjugates Using Size Exclusion Chromatography

In total, 25 μg of the respective conjugate in 100 μl were injected onto a Superdex Increase 200 10/30 GL gel filtration column (GE Healthcare, #28989336) coupled to an Agilent 1200 series HPLC system. SEC runs were performed at a 0.5 ml/min flow rate with PBS as a running buffer. Proteins were detected by an online 280 nm absorption measurement. Data analysis and peak integrations were performed using GraphPad Prism 8.0.

### Results

### Western Blot and Biolayer Interferometry of Expressed Conjugates

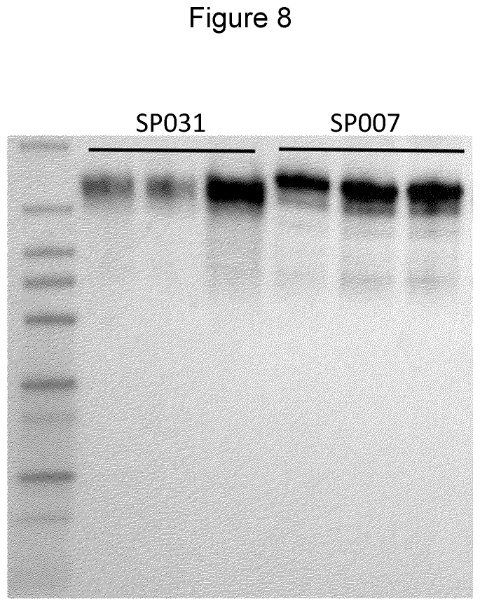

The results of western blot of the 2.5 ml cultures of SP007 and SP031 are shown in FIG. 8. Stronger intensities were obtained for SP007 than for SP031. Overall, the western blot indicated higher titers for the construct comprising a humanized scFv, SP007, versus the construct comprising a mouse scFv, SP031.

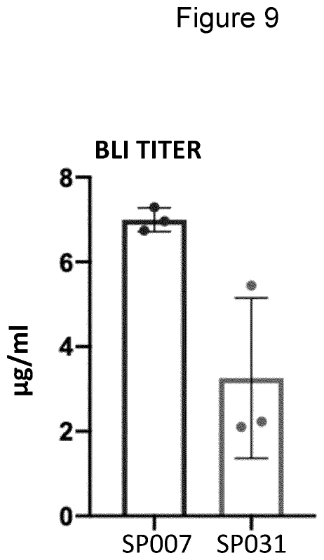

Biolayer interferometry titer measurements on supernatants 5 days post transfection are shown in FIG. 9. The titers for SP007 were appr 3-fold higher than those for SP031, mirroring the results observed from the western blot.

### Purity Measurements of Conjugates Using Size Exclusion Chromatography

Expression and purification of the SP031 conjugate resulted in a monomeric population which was clearly below the quality control cut-off for monomeric content set to 95% as judged by SEC. Analysis of the chromatogram for SP031 resulted in a 85.3% monomeric content after affinity purification, with a distinct peak representing a smaller, non-native population of lower molecular species making up 14.7% of the total sample. All other tested conjugates (SP007, SP019 and SP027), comprising humanized scFv:s, displayed superior SEC profiles after purification compared to the ancestral mouse version (Table 5).

TABLE 5
Monomeric content of conjugates

| Construct | Monomeric content (%) |
|---|---|
| SP031 | 85.3 |
| SP007 | 95.0 |
| SP019 | 99.2 |
| SP027 | 95.6 |

### Conclusion

The construct comprising the mouse scFv IBIIICI (variant SP031) showed significantly lower expression levels compared to constructs comprising humanized scFv:s. Furthermore, the SEC profile of SP031 exhibited a considerable amount of undesired species, with a total monomeric population of 85.3%. From a developability perspective, an improved profile was observed for all conjugates comprising the humanized scFv:SP007, SP019 and SP027 with a resulting >95% monomeric population observed in the SEC results. Thus, the conjugates comprising humanized scFv together with anti-CD40 antibody were deemed superior from a developability perspective as compared to conjugates comprising the non-humanized scFv.

### Example 7

### Efficacy of Tumor Treatment

The anti-tumor responses induced by repeated vaccination with peptides in combination with the bispecific binding conjugate SP027 in the TC-1 tumor model were investigated. By local vaccination at a non-tumor site, tag constructs comprising a tumor-associated antigen (of viral or neoantigen origin) were delivered to dendritic cells by the bispecific conjugate's binding to CD40 and internalized to release the antigenic peptides. The antigen peptides were processed and presented to T cells. With repeated injections, antigen-specific T cells expand, migrate and target tumor cells. The anti-tumor effect of the complex of bispecific conjugate and tag construct was compared to the peptide alone and/or to the bispecific conjugate alone, also injected at a non-tumor site.

### Materials and Methods

Mice, strain B-hCD40 (Biocytogen) (9-12 weeks) were divided into the following three treatment groups (6-7 mice per group): Group 1: a high dose of 30 μg antigenic peptide UU0171 (SEQ ID NO:77; immunogenic E7 derived synthetic long peptide HPV16 E7 [HPV16 E7 44-62]); Group 2: bispecific binding conjugate SP027 alone, at 50 μg in first injection on day 5 and at 30 μg in injections on day 10 and 15; Group 3: bispecific binding conjugate SP027 at 50 μg in first injection on day 5 and at 30 μg in injections on day 10 and 15 in complex with tag construct UU0169 (SEQ ID NO:78) comprising a 9 aa tag moiety linked to the HPV peptide and given at a low dose of 3 μg in the complex mixture at every dosing. Appr 5×10⁵ tumor cells were injected s.c. in the right flank at day 0. Injection of treatment to the mice was performed s.c. in the left hock on day 5, day 10 and day 15, i.e. therapy was not given at the site of tumor growth. Tumor growth and survival were monitored 3 times a week, and the mice are sacrificed when the tumor size reached a maximum of 1000 mm³ as the experimental endpoint, or if reaching the humane endpoint (e.g. health status with regard to wounds, weight and appearance. Mice reaching the humane endpoint while not reaching the experimental endpoint were censored.

### Results

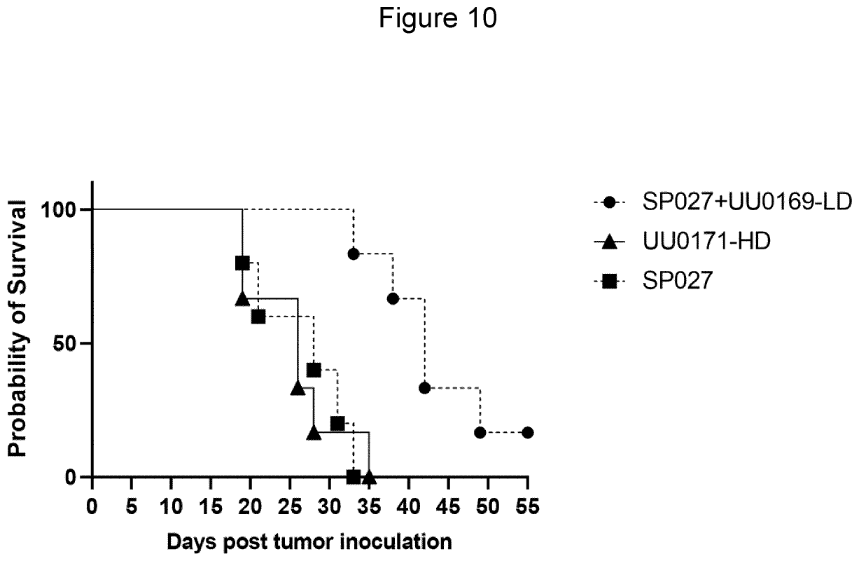

The results are shown in FIG. 10. The TC-1 peptide from the oncogenic HPV protein E7 coupled to a 9-mer tag moiety (UU0169) and delivered in complex with the bispecific conjugate construct SP027 induced a potent immune response and reduced the tumor volume significantly compared to the control groups that were given either the bispecific conjugate SP027 alone or a high dose of the antigenic peptide without tag moiety (UU0171), even though the therapy was injected at a non-tumor site at a low dose.

Informal Sequence Listing
SEQ ID NO: 1
QVQLVQSGAEVKKPGASVKVSCKASGFNIKDFNIHWVRQAPGQGL
EWIGRIDPESGDAEYVPKFQGRVTMTRDTSTSTVYMELSSLRSED
TAVYYCTTGSYDLDVEYWGQGTLVTVSS
SEQ ID NO: 2
QVQLVQSGAEVKKPGASVKVSCKASGFNIKDFNIHWVRQAPGQGL
EWIGRIDPEGGDAEYVPKFQGRVTMTRDTSTSTVYMELSSLRSED
TAVYYCTTGSYDLDVEYWGQGTLVTVSS
SEQ ID NO: 3
QVQLVQSGAEVKKPGASVKVSCKASGFNIKDFNIHWVRQAPGQGL
EWIGRIDPEQGDAEYVPKFQGRVTMTRDTSTSTVYMELSSLRSED
TAVYYCTTGSYDLDVEYWGQGTLVTVSS
SEQ ID NO: 4
QVQLVQSGAEVKKPGASVKVSCKASGFNIKDFNIHWVRQAPGQGL
EWIGRIDPEDGDAEYVPKFQGRVTMTRDTSTSTVYMELSSLRSED
TAVYYCTTGSYDLDVEYWGQGTLVTVSS
SEQ ID NO: 5
QVQLVQSGAEVKKPGASVKVSCKASGFNIKDFNIHWVRQAPGQGL
EWIGRIDPENADAEYVPKFQGRVTMTRDTSTSTVYMELSSLRSED
TAVYYCTTGSYDLDVEYWGQGTLVTVSS
SEQ ID NO: 6
QVQLVQSGAEVKKPGASVKVSCKASGFNIKDFNIHWVRQAPGQGL
EWIGRIDPENGDAEYVPKFQGRVTMTRDTSTSTVYMELSSLRSED
TAVYYCTTGSYDLDVEYWGQGTLVTVSS
SEQ ID NO: 7
FNIKDFNI
SEQ ID NO: 8)
IGRIDPEXₐX_{b}DAEYVP
SEQ ID NO: 9
TTGSYDLDVE
SEQ ID NO: 10
DIQMTQSPSSLSASVGDRVTITCHASQNINVWLSWYQQKPGKAPK
LLIYKASTLHTGVPSRFSGSGSGTDFTLTISSLQPEDFATYYCQQ
GQSYPLTFGQGTKLEIK
SEQ ID NO: 11
HASQNINVWLS
SEQ ID NO: 12
KASTLHT
SEQ ID NO: 13
QQGQSYPLT
SEQ ID NO: 14
GGGGSGGGGSGGGGSGGGGS
SEQ ID NO: 15
QSISSY
SEQ ID NO: 16
AAS
SEQ ID NO: 17
QQGYPYPFT
SEQ ID NO: 18
GFTFSSYA
SEQ ID NO: 19
ISGYSGST
SEQ ID NO: 20
ARYYSYYGYYYFDY
SEQ ID NO: 21
DIQMTQSPSSLSASVGDRVTITCRASQSISSYLNWYQQKPGKAPK
LLIYAASSLQSGVPSRFSGSGSGTDFTLTISSLQPEDFATYYCQQ
GYPYPFTFGQGTKLEIK
SEQ ID NO: 22
EVQLLESGGGLVQPGGSLRLSCAASGFTFSSYAMSWVRQAPGKGL
EWVSGISGYSGSTYYADSVKGRFTISRDNSKNTLYLQMNSLRAED
TAVYYCARYYSYYGYYYFDYWGQGTLVTVSS
SEQ ID NO: 23
DIQMTQSPSSLSASVGDRVTITCRASQSISSYLNWYQQKPGKAPK
LLIYAASSLQSGVPSRFSGSGSGTDFTLTISSLQPEDFATYYCQQ
GYPYPFTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 24
EVQLLESGGGLVQPGGSLRLSCAASGFTFSSYAMSWVRQAPGKGL
EWVSGISGYSGSTYYADSVKGRFTISRDNSKNTLYLQMNSLRAED
TAVYYCARYYSYYGYYYFDYWGQGTLVTVSSASTKGPSVFPLAPC
SRSTSESTAALGCLVKDYFPEPVTVSWNSGALTSGVHTFPAVLQS
SGLYSLSSVVTVPSSNFGTQTYTCNVDHKPSNTKVDKTVERKCCV
ECPPCPAPPVAGPSVFLFPPKPKDTLMISRTPEVTCVVVDVSHED
PEVQFNWYVDGVEVHNAKTKPREEQFNSTFRVVSVLTVVHQDWLN
GKEYKCKVSNKGLPAPIEKTISKTKGQPREPQVYTLPPSREEMTK
NQVSLTCLVKGFYPSDIAVEWESNGQPENNYKTTPPMLDSDGSFF
LYSKLTVDKSRWQQGNVFSCSVMHEALHNHYTQKSLSLSPGK
SEQ ID NO: 25
EVRLLQSGAALVRPGASVKLSCTASGFNIKDFNIHWVKQRPEQGL
EWIGRIDPENGDAEYVPKFQVRATMTTDTSSNTVYLHLSSLTSGD
TAVYYCTTGSYDLDVEYWGQGTTLTVSSGGGGSGGGGSGGGGSGG
GGSELQMTQSPSSLSASLGDTVTITCHASQNINVWLSWYQQRPGN
IPKLLIYKASTLHTGVPSRFRGSGSGTGFTLTISSLQPEDIATYY
CQQGQSYPLTFGAGTKLELK
SEQ ID NO: 26
FIGITELKKLESKINKVFK-Biotin
SEQ ID NO: 27
FIGITELKKLESKINK
SEQ ID NO: 28
FIGITELKKLESKIN
SEQ ID NO: 29
FIGITELKKLESKI
SEQ ID NO: 30
FIGITELKKLESK
SEQ ID NO: 31
FIGITELKKLES
SEQ ID NO: 32
LEQLESIINFEKLLAAAAAKFIGITELKKLES
SEQ ID NO: 33
FIGITELKKLESLEQLESIINFEKLLAAAAAK
SEQ ID NO: 34
Biotin-(PEG3)FIGITELKKLES
SEQ ID NO: 35
FIGITELKKLES(PEG3)K-Biotin
SEQ ID NO: 36
FIGITELHHLESK-Biotin
SEQ ID NO: 37
FIGISELKKLESK-Biotin
SEQ ID NO: 38
FIGITELHKLESK-Biotin
SEQ ID NO: 39
FIGITELKHLESK-Biotin
SEQ ID NO: 40
FIGITELKKLE
SEQ ID NO: 41
FIGITELKKL
SEQ ID NO: 42
FIGITELKK
SEQ ID NO: 43
FIGITELK
SEQ ID NO: 44
FIGITELLEQLESIINFEKLAAAAAK
SEQ ID NO: 45
FIGITELKLEQLESIINFEKLAAAAAK
SEQ ID NO: 46
FIGITELHLEQLESIINFEKLAAAAAK
SEQ ID NO: 47
ANSKFIGITELK
SEQ ID NO: 48
Biotin-IDIKNDLYEKTLNDYKAIANKLSQV
SEQ ID NO: 49
QVQLVQSGAEVKKPGASVKVSCKASGFNIKDFNIHWVRQAPGQGL
EWIGRIDPESGDAEYVPKFQGRVTMTRDTSTSTVYMELSSLRSED
TAVYYCTTGSYDLDVEYWGQGTLVTVSSGGGGGGGGSGGGGSGGG
GSDIQMTQSPSSLSASVGDRVTITCHASQNINVWLSWYQQKPGKA
PKLLIYKASTLHTGVPSRFSGSGSGTDFTLTISSLQPEDFATYYC
QQGQSYPLTFGQGTKLEIK
SEQ ID NO: 50
QVQLVQSGAEVKKPGASVKVSCKASGFNIKDFNIHWVRQAPGQGL
EWIGRIDPEGGDAEYVPKFQGRVTMTRDTSTSTVYMELSSLRSED
TAVYYCTTGSYDLDVEYWGQGTLVTVSSGGGGSGGGGSGGGGSGG
GGSDIQMTQSPSSLSASVGDRVTITCHASQNINVWLSWYQQKPGK
APKLLIYKASTLHTGVPSRFSGSGSGTDFTLTISSLQPEDFATYY
CQQGQSYPLTFGQGTKLEIK
SEQ ID NO: 51
QVQLVQSGAEVKKPGASVKVSCKASGFNIKDFNIHWVRQAPGQGL
EWIGRIDPEQGDAEYVPKFQGRVTMTRDTSTSTVYMELSSLRSED
TAVYYCTTGSYDLDVEYWGQGTLVTVSSGGGGSGGGGSGGGGGGG
GSDIQMTQSPSSLSASVGDRVTITCHASQNINVWLSWYQQKPGKA
PKLLIYKASTLHTGVPSRFSGSGSGTDFTLTISSLQPEDFATYYC
QQGQSYPLTFGQGTKLEIK
SEQ ID NO: 52
QVQLVQSGAEVKKPGASVKVSCKASGFNIKDFNIHWVRQAPGQGL
EWIGRIDPEDGDAEYVPKFQGRVTMTRDTSTSTVYMELSSLRSED
TAVYYCTTGSYDLDVEYWGQGTLVTVSSGGGGSGGGGSGGGGSGG
GGSDIQMTQSPSSLSASVGDRVTITCHASQNINVWLSWYQQKPGK
APKLLIYKASTLHTGVPSRFSGSGSGTDFTLTISSLQPEDFATYY
CQQGQSYPLTFGQGTKLEIK
SEQ ID NO: 53
QVQLVQSGAEVKKPGASVKVSCKASGFNIKDFNIHWVRQAPGQGL
EWIGRIDPENADAEYVPKFQGRVTMTRDTSTSTVYMELSSLRSED
TAVYYCTTGSYDLDVEYWGQGTLVTVSSGGGGSGGGGSGGGGSGG
GGSDIQMTQSPSSLSASVGDRVTITCHASQNINVWLSWYQQKPGK
APKLLIYKASTLHTGVPSRFSGSGSGTDFTLTISSLQPEDFATYY
CQQGQSYPLTFGQGTKLEIK
SEQ ID NO: 54
QVQLVQSGAEVKKPGASVKVSCKASGFNIKDFNIHWVRQAPGQGL
EWIGRIDPENGDAEYVPKFQGRVTMTRDTSTSTVYMELSSLRSED
TAVYYCTTGSYDLDVEYWGQGTLVTVSSGGGGSGGGGSGGGGSGG
GGSDIQMTQSPSSLSASVGDRVTITCHASQNINVWLSWYQQKPGK
APKLLIYKASTLHTGVPSRFSGSGSGTDFTLTISSLQPEDFATYY
CQQGQSYPLTFGQGTKLEIK
SEQ ID NO: 55
EVRLLQSGAALVRPGASVKLSCTASGFNIKDFNIHWVKQRPEQGL
EWIGRIDPENGDAEYVPKFQVRATMTTDTSSNTVYLHLSSLTSGD
TAVYYCTTGSYDLDVEYWGQGTTLTVSS
SEQ ID NO: 56
ELQMTQSPSSLSASLGDTVTITCHASQNINVWLSWYQQRPGNIPK
LLIYKASTLHTGVPSRFRGSGSGTGFTLTISSLQPEDIATYYCQQ
GQSYPLTFGAGTKLELK
SEQ ID NO: 57
EVQLLESGGGLVQPGGSLRLSCAASGFTFSSYAMSWVRQAPGKGL
EWVSGISGYSGSTYYADSVKGRFTISRDNSKNTLYLQMNSLRAED
TAVYYCARYYSYYGYYYFDYWGQGTLVTVSSASTKGPSVFPLAPC
SRSTSESTAALGCLVKDYFPEPVTVSWNSGALTSGVHTFPAVLQS
SGLYSLSSVVTVPSSNFGTQTYTCNVDHKPSNTKVDKTVERKCCV
ECPPCPAPPVAGPSVFLFPPKPKDTLMISRTPEVTCVVVDVSHED
PEVQFNWYVDGVEVHNAKTKPREEQFNSTFRVVSVLTVVHQDWLN
GKEYKCKVSNKGLPAPIEKTISKTKGQPREPQVYTLPPSREEMTK
NQVSLTCLVKGFYPSDIAVEWESNGQPENNYKTTPPMLDSDGSFF
LYSKLTVDKSRWQQGNVFSCSVMHEALHNHYTQKSLSLSPGKGGG
GSGGGGSEVRLLQSGAALVRPGASVKLSCTASGFNIKDFNIHWVK
QRPEQGLEWIGRIDPENGDAEYVPKFQVRATMTTDTSSNTVYLHL
SSLTSGDTAVYYCTTGSYDLDVEYWGQGTTLTVSSGGGGSGGGGS
GGGGSGGGGSELQMTQSPSSLSASLGDTVTITCHASQNINVWLSW
YQQRPGNIPKLLIYKASTLHTGVPSRFRGSGSGTGFTLTISSLQP
EDIATYYCQQGQSYPLTFGAGTKLELK
SEQ ID NO: 58
DIQMTQSPSSLSASVGDRVTITCRASQSISSYLNWYQQKPGKAPK
LLIYAASSLQSGVPSRFSGSGSGTDFTLTISSLQPEDFATYYCQQ
GYPYPFTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 59
EVQLLESGGGLVQPGGSLRLSCAASGFTFSSYAMSWVRQAPGKGL
EWVSGISGYSGSTYYADSVKGRFTISRDNSKNTLYLQMNSLRAED
TAVYYCARYYSYYGYYYFDYWGQGTLVTVSSASTKGPSVFPLAPC
SRSTSESTAALGCLVKDYFPEPVTVSWNSGALTSGVHTFPAVLQS
SGLYSLSSVVTVPSSNFGTQTYTCNVDHKPSNTKVDKTVERKCCV
ECPPCPAPPVAGPSVFLFPPKPKDTLMISRTPEVTCVVVDVSHED
PEVQFNWYVDGVEVHNAKTKPREEQFNSTFRVVSVLTVVHQDWLN
GKEYKCKVSNKGLPAPIEKTISKTKGQPREPQVYTLPPSREEMTK
NQVSLTCLVKGFYPSDIAVEWESNGQPENNYKTTPPMLDSDGSFF
LYSKLTVDKSRWQQGNVFSCSVMHEALHNHYTQKSLSLSPGKGGG
GSGGGGSQVQLVQSGAEVKKPGASVKVSCKASGFNIKDFNIHWVR
QAPGQGLEWIGRIDPENGDAEYVPKFQGRVTMTRDTSTSTVYMEL
SSLRSEDTAVYYCTTGSYDLDVEYWGQGTLVTVSSGGGGSGGGGS
GGGGSGGGGSDIQMTQSPSSLSASVGDRVTITCHASQNINVWLSW
YQQKPGKAPKLLIYKASTLHTGVPSRFSGSGSGTDFTLTISSLQP
EDFATYYCQQGQSYPLTFGQGTKLEIK
SEQ ID NO: 60
EVQLLESGGGLVQPGGSLRLSCAASGFTFSSYAMSWVRQAPGKGL
EWVSGISGYSGSTYYADSVKGRFTISRDNSKNTLYLQMNSLRAED
TAVYYCARYYSYYGYYYFDYWGQGTLVTVSSASTKGPSVFPLAPC
SRSTSESTAALGCLVKDYFPEPVTVSWNSGALTSGVHTFPAVLQS
SGLYSLSSVVTVPSSNFGTQTYTCNVDHKPSNTKVDKTVERKCCV
ECPPCPAPPVAGPSVFLFPPKPKDTLMISRTPEVTCVVVDVSHED
PEVQFNWYVDGVEVHNAKTKPREEQFNSTFRVVSVLTVVHQDWLN
GKEYKCKVSNKGLPAPIEKTISKTKGQPREPQVYTLPPSREEMTK
NQVSLTCLVKGFYPSDIAVEWESNGQPENNYKTTPPMLDSDGSFF
LYSKLTVDKSRWQQGNVFSCSVMHEALHNHYTQKSLSLSPGKGGG
GSGGGGSQVQLVQSGAEVKKPGASVKVSCKASGFNIKDFNIHWVR
QAPGQGLEWIGRIDPESGDAEYVPKFQGRVTMTRDTSTSTVYMEL
SSLRSEDTAVYYCTTGSYDLDVEYWGQGTLVTVSSGGGGSGGGGS
GGGGSGGGGSDIQMTQSPSSLSASVGDRVTITCHASQNINVWLSW
YQQKPGKAPKLLIYKASTLHTGVPSRFSGSGSGTDFTLTISSLQP
EDFATYYCQQGQSYPLTFGQGTKLEIK
SEQ ID NO: 61
DIQMTQSPSSLSASVGDRVTITCRASQSISSYLNWYQQKPGKAPK
LLIYAASFLQSGVPSRFSGSGSGTDFTLTISSLQPEDFATYYCQQ
GYPYPFTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 62
IGRIDPESGDAEYVP
SEQ ID NO: 63
IGRIDPEGGDAEYVP
SEQ ID NO: 64
IGRIDPEQGDAEYVP
SEQ ID NO: 65
IGRIDPEDGDAEYVP
SEQ ID NO: 66
IGRIDPENADAEYVP
SEQ ID NO: 67
IGRIDPENGDAEYVP
SEQ ID NO: 68
QVQLQQPGAELVMPGASVNLSCKASGYTFTDYWMHWVKQRPGQGL
EWIGEIDPSDNFSNLNQNFRGKATLTVDKSSRTAFLQLSSLTSED
SAVYYCAVEDYWGQGTTLTVSS
SEQ ID NO: 69
SDIVMTQATPSVLVTPGEAVSISCRASRSLLHSNGITYLYWFLQR
PGQSPQVLIYRMSNLVSGVPDRFSGSGSGTAFTLRISRVEAEDVG
VYYCMQHLEFPYTFGGGTKLEIK
SEQ ID NO: 70
GGGGS
SEQ ID NO: 71
GGGGSGGGGS
SEQ ID NO: 72
GGGGSGGGGSGGGGS
SEQ ID NO: 73
GGGGSGGGGSGGGGSGGGGSGGGGS
SEQ ID NO: 74
FIGITELL
SEQ ID NO: 75
FIGITELH
SEQ ID NO: 76
FIGISELK
SEQ ID NO: 77
QAEPDRAHYNIVTFCCKCD
SEQ ID NO: 78
FIGITELKKQAEPDRAHYNIVTFCCKCD
SEQ ID NO: 79
FIGITELLK
SEQ ID NO: 80
FIGITELHK

### ITEMIZED LISTING OF EMBODIMENTS

1. A binding molecule, comprising:
   an immunoglobulin heavy chain variable region (VH) consisting of an amino acid sequence selected from the group consisting of SEQ ID NO:1-6 and amino acid sequences having at least 90% identity thereto, provided that said heavy chain variable region (VH) comprises three complementarity determining domains (CDRs), wherein:
      VHCDR1 has the sequence set forth in SEQ ID NO:7;
      VHCDR2 has the sequence IGRIDPEXₐX_{b}DAEYVP (SEQ ID NO:8), wherein XₐX_{b} is selected from the group consisting of SG, GG, QG, DG, NA and NG; and
      VHCDR3 has the sequence set forth in SEQ ID NO:9; and
   an immunoglobulin light chain variable region (VL) consisting of an amino acid sequence selected from the group consisting of SEQ ID NO:10 and amino acid sequences having at least 90% identity thereto, provided that said light chain variable region (VL) comprises three complementarity determining domains (CDRs), wherein:
      VLCDR1 has the sequence set forth in SEQ ID NO:11;
      VLCDR2 has the sequence set forth in SEQ ID NO:12; and
      VLCDR3 has the sequence set forth in SEQ ID NO:13.
2. Binding molecule according to item 1, wherein said heavy chain variable region (VH) consists of an amino acid sequence selected from the group consisting of SEQ ID NO:2-6 and amino acid sequences having at least 90% identity thereto.
3. Binding molecule according to item 1, wherein said heavy chain variable region (VH) consists of an amino acid sequence selected from the group consisting of SEQ ID NO:1, 3, 4, 5 and 6 and amino acid sequences having at least 90% identity thereto.
4. Binding molecule according to item 1, wherein said heavy chain variable region (VH) consists of an amino acid sequence selected from the group consisting of SEQ ID NO:1, 2, 4, 5 and 6 and amino acid sequences having at least 90% identity thereto.
5. Binding molecule according to item 1, wherein said heavy chain variable region (VH) consists of an amino acid sequence selected from the group consisting of SEQ ID NO:1, 2, 3, 5 and 6 and amino acid sequences having at least 90% identity thereto.
6. Binding molecule according to item 1, wherein said heavy chain variable region (VH) consists of an amino acid sequence selected from the group consisting of SEQ ID NO:1, 2, 3, 4 and 6 and amino acid sequences having at least 90% identity thereto.
7. Binding molecule according to item 1, wherein said heavy chain variable region (VH) consists of an amino acid sequence selected from the group consisting of SEQ ID NO:1-5 and amino acid sequences having at least 90% identity thereto.
8. Binding molecule according to any one of items 2-3, wherein said heavy chain variable region (VH) consists of an amino acid sequence selected from the group consisting of SEQ ID NO:3-6 and amino acid sequences having at least 90% identity thereto.
9. Binding molecule according to any one of items 2 and 4, wherein said heavy chain variable region (VH) consists of an amino acid sequence selected from the group consisting of SEQ ID NO:2, 4, 5 and 6 and amino acid sequences having at least 90% identity thereto.
10. Binding molecule according to any one of items 2 and 5, wherein said heavy chain variable region (VH) consists of an amino acid sequence selected from the group consisting of SEQ ID NO:2, 3, 5 and 6 and amino acid sequences having at least 90% identity thereto.
11. Binding molecule according to any one of items 2 and 6, wherein said heavy chain variable region (VH) consists of an amino acid sequence selected from the group consisting of SEQ ID NO:2, 3, 4 and 6 and amino acid sequences having at least 90% identity thereto.
12. Binding molecule according to any one of items 2 and 7, wherein said heavy chain variable region (VH) consists of an amino acid sequence selected from the group consisting of SEQ ID NO:2-5 and amino acid sequences having at least 90% identity thereto.
13. Binding molecule according to any one of items 3 and 4, wherein said heavy chain variable region (VH) consists of an amino acid sequence selected from the group consisting of SEQ ID NO:1, 4, 5 and 6 and amino acid sequences having at least 90% identity thereto.
14. Binding molecule according to any one of items 3 and 5, wherein said heavy chain variable region (VH) consists of an amino acid sequence selected from the group consisting of SEQ ID NO:1, 3, 5 and 6 and amino acid sequences having at least 90% identity thereto.
15. Binding molecule according to any one of items 3 and 6, wherein said heavy chain variable region (VH) consists of an amino acid sequence selected from the group consisting of SEQ ID NO:1, 3, 4 and 6 and amino acid sequences having at least 90% identity thereto.
16. Binding molecule according to any one of items 3 and 7, wherein said heavy chain variable region (VH) consists of an amino acid sequence selected from the group consisting of SEQ ID NO:1, 3, 4 and 5 and amino acid sequences having at least 90% identity thereto.
17. Binding molecule according to any one of items 4 and 5, wherein said heavy chain variable region (VH) consists of an amino acid sequence selected from the group consisting of SEQ ID NO:1, 2, 5 and 6 and amino acid sequences having at least 90% identity thereto.
18. Binding molecule according to any one of items 4 and 6, wherein said heavy chain variable region (VH) consists of an amino acid sequence selected from the group consisting of SEQ ID NO:1, 2, 4 and 6 and amino acid sequences having at least 90% identity thereto.
19. Binding molecule according to any one of items 4 and 7, wherein said heavy chain variable region (VH) consists of an amino acid sequence selected from the group consisting of SEQ ID NO:1, 2, 4 and 5 and amino acid sequences having at least 90% identity thereto.
20. Binding molecule according to any one of items 5 and 6, wherein said heavy chain variable region (VH) consists of an amino acid sequence selected from the group consisting of SEQ ID NO:1, 2, 3 and 6 and amino acid sequences having at least 90% identity thereto.
21. Binding molecule according to any one of items 5 and 7, wherein said heavy chain variable region (VH) consists of an amino acid sequence selected from the group consisting of SEQ ID NO:1, 2, 3 and 5 and amino acid sequences having at least 90% identity thereto.
22. Binding molecule according to any one of items 6 and 7, wherein said heavy chain variable region (VH) consists of an amino acid sequence selected from the group consisting of SEQ ID NO:1-4 and amino acid sequences having at least 90% identity thereto.
23. Binding molecule according to any one of items 8, 9 and 13, wherein said heavy chain variable region (VH) consists of an amino acid sequence selected from the group consisting of SEQ ID NO:4-6 and amino acid sequences having at least 90% identity thereto.
24. Binding molecule according to any one of items 8, 10 and 14, wherein said heavy chain variable region (VH) consists of an amino acid sequence selected from the group consisting of SEQ ID NO:3, 5 and 6 and amino acid sequences having at least 90% identity thereto.
25. Binding molecule according to any one of items 8, 11 and 15, wherein said heavy chain variable region (VH) consists of an amino acid sequence selected from the group consisting of SEQ ID NO:3, 4 and 6 and amino acid sequences having at least 90% identity thereto.
26. Binding molecule according to any one of items 8, 12 and 16, wherein said heavy chain variable region (VH) consists of an amino acid sequence selected from the group consisting of SEQ ID NO:3, 4 and 5 and amino acid sequences having at least 90% identity thereto.
27. Binding molecule according to any one of items 9, 10 and 17, wherein said heavy chain variable region (VH) consists of an amino acid sequence selected from the group consisting of SEQ ID NO:2, 5 and 6 and amino acid sequences having at least 90% identity thereto.
28. Binding molecule according to any one of items 9, 11 and 18, wherein said heavy chain variable region (VH) consists of an amino acid sequence selected from the group consisting of SEQ ID NO:2, 4 and 6 and amino acid sequences having at least 90% identity thereto.
29. Binding molecule according to any one of items 9, 12 and 19, wherein said heavy chain variable region (VH) consists of an amino acid sequence selected from the group consisting of SEQ ID NO:2, 4 and 5 and amino acid sequences having at least 90% identity thereto.
30. Binding molecule according to any one of items 10, 11 and 20, wherein said heavy chain variable region (VH) consists of an amino acid sequence selected from the group consisting of SEQ ID NO:2, 3 and 6 and amino acid sequences having at least 90% identity thereto.
31. Binding molecule according to any one of items 10, 12 and 21, wherein said heavy chain variable region (VH) consists of an amino acid sequence selected from the group consisting of SEQ ID NO:2, 3 and 5 and amino acid sequences having at least 90% identity thereto.
32. Binding molecule according to any one of items 11, 12 and 22, wherein said heavy chain variable region (VH) consists of an amino acid sequence selected from the group consisting of SEQ ID NO:2-4 and amino acid sequences having at least 90% identity thereto.
33. Binding molecule according to any one of items 13, 14 and 17, wherein said heavy chain variable region (VH) consists of an amino acid sequence selected from the group consisting of SEQ ID NO:1, 5 and 6 and amino acid sequences having at least 90% identity thereto.
34. Binding molecule according to any one of items 13, 15 and 18, wherein said heavy chain variable region (VH) consists of an amino acid sequence selected from the group consisting of SEQ ID NO:1, 4 and 6 and amino acid sequences having at least 90% identity thereto.
35. Binding molecule according to any one of items 13, 16 and 19, wherein said heavy chain variable region (VH) consists of an amino acid sequence selected from the group consisting of SEQ ID NO:1, 4 and 5 and amino acid sequences having at least 90% identity thereto.
36. Binding molecule according to any one of items 14, 15 and 20, wherein said heavy chain variable region (VH) consists of an amino acid sequence selected from the group consisting of SEQ ID NO:1, 3 and 6 and amino acid sequences having at least 90% identity thereto.
37. Binding molecule according to any one of items 14, 16 and 21, wherein said heavy chain variable region (VH) consists of an amino acid sequence selected from the group consisting of SEQ ID NO:1, 3 and 5 and amino acid sequences having at least 90% identity thereto.
38. Binding molecule according to any one of items 15, 16 and 22, wherein said heavy chain variable region (VH) consists of an amino acid sequence selected from the group consisting of SEQ ID NO:1, 3 and 4 and amino acid sequences having at least 90% identity thereto.
39. Binding molecule according to any one of items 17, 18 and 20, wherein said heavy chain variable region (VH) consists of an amino acid sequence selected from the group consisting of SEQ ID NO:1, 2 and 6 and amino acid sequences having at least 90% identity thereto.
40. Binding molecule according to any one of items 17, 19 and 21, wherein said heavy chain variable region (VH) consists of an amino acid sequence selected from the group consisting of SEQ ID NO:1, 2 and 5 and amino acid sequences having at least 90% identity thereto.
41. Binding molecule according to any one of items 18, 19 and 22, wherein said heavy chain variable region (VH) consists of an amino acid sequence selected from the group consisting of SEQ ID NO:1, 2 and 4 and amino acid sequences having at least 90% identity thereto.
42. Binding molecule according to any one of items 18, 19 and 22, wherein said heavy chain variable region (VH) consists of an amino acid sequence selected from the group consisting of SEQ ID NO:1-3 and amino acid sequences having at least 90% identity thereto.
43. Binding molecule according to any one of items 23, 24, 27 and 33, wherein said heavy chain variable region (VH) consists of an amino acid sequence selected from the group consisting of SEQ ID NO:5-6 and amino acid sequences having at least 90% identity thereto.
44. Binding molecule according to any one of items 23, 25, 28 and 34, wherein said heavy chain variable region (VH) consists of an amino acid sequence selected from the group consisting of SEQ ID NO:4 and 6 and amino acid sequences having at least 90% identity thereto.
45. Binding molecule according to any one of items 23, 26, 29 and 35, wherein said heavy chain variable region (VH) consists of an amino acid sequence selected from the group consisting of SEQ ID NO:4-5 and amino acid sequences having at least 90% identity thereto.
46. Binding molecule according to any one of items 24, 25, 30 and 36, wherein said heavy chain variable region (VH) consists of an amino acid sequence selected from the group consisting of SEQ ID NO:3 and 6 and amino acid sequences having at least 90% identity thereto.
47. Binding molecule according to any one of items 24, 26, 31 and 37, wherein said heavy chain variable region (VH) consists of an amino acid sequence selected from the group consisting of SEQ ID NO:3 and 5 and amino acid sequences having at least 90% identity thereto.
48. Binding molecule according to any one of items 25, 26, 32 and 38, wherein said heavy chain variable region (VH) consists of an amino acid sequence selected from the group consisting of SEQ ID NO:3-4 and amino acid sequences having at least 90% identity thereto.
49. Binding molecule according to any one of items 27, 28, 30 and 39, wherein said heavy chain variable region (VH) consists of an amino acid sequence selected from the group consisting of SEQ ID NO:2 and 6 and amino acid sequences having at least 90% identity thereto.
50. Binding molecule according to any one of items 27, 29, 31 and 40, wherein said heavy chain variable region (VH) consists of an amino acid sequence selected from the group consisting of SEQ ID NO:2 and 5 and amino acid sequences having at least 90% identity thereto.
51. Binding molecule according to any one of items 28, 29, 32 and 41, wherein said heavy chain variable region (VH) consists of an amino acid sequence selected from the group consisting of SEQ ID NO:2 and 4 and amino acid sequences having at least 90% identity thereto.
52. Binding molecule according to any one of items 30, 31, 32 and 42, wherein said heavy chain variable region (VH) consists of an amino acid sequence selected from the group consisting of SEQ ID NO:2-3 and amino acid sequences having at least 90% identity thereto.
53. Binding molecule according to any one of items 33, 34, 36 and 39, wherein said heavy chain variable region (VH) consists of an amino acid sequence selected from the group consisting of SEQ ID NO:1 and 6 and amino acid sequences having at least 90% identity thereto.
54. Binding molecule according to any one of items 33, 35, 37 and 40, wherein said heavy chain variable region (VH) consists of an amino acid sequence selected from the group consisting of SEQ ID NO:1 and 5 and amino acid sequences having at least 90% identity thereto.
55. Binding molecule according to any one of items 34, 35, 38 and 41, wherein said heavy chain variable region (VH) consists of an amino acid sequence selected from the group consisting of SEQ ID NO:1 and 4 and amino acid sequences having at least 90% identity thereto.
56. Binding molecule according to any one of items 36, 37, 38 and 42, wherein said heavy chain variable region (VH) consists of an amino acid sequence selected from the group consisting of SEQ ID NO:1 and 3 and amino acid sequences having at least 90% identity thereto.
57. Binding molecule according to any one of items 39-42, wherein said heavy chain variable region (VH) consists of an amino acid sequence selected from the group consisting of SEQ ID NO:1-2 and amino acid sequences having at least 90% identity thereto.
58. Binding molecule according to any one of items 43, 44, 46, 49 and 53, wherein said heavy chain variable region (VH) consists of the amino acid sequence SEQ ID NO:6 and amino acid sequences having at least 90% identity thereto.
59. Binding molecule according to any one of items 43, 45, 47, 50 and 54, wherein said heavy chain variable region (VH) consists of the amino acid sequence SEQ ID NO:5 and amino acid sequences having at least 90% identity thereto.
60. Binding molecule according to any one of items 44, 45, 48, 51 and 55, wherein said heavy chain variable region (VH) consists of the amino acid sequence SEQ ID NO:4 and amino acid sequences having at least 90% identity thereto.
61. Binding molecule according to any one of items 46-48, 52 and 56, wherein said heavy chain variable region (VH) consists of the amino acid sequence SEQ ID NO: 3 and amino acid sequences having at least 90% identity thereto.
62. Binding molecule according to any one of items 49-52 and 57, wherein said heavy chain variable region (VH) consists of the amino acid sequence SEQ ID NO: 2 and amino acid sequences having at least 90% identity thereto.
63. Binding molecule according to any one of items 53-57, wherein said heavy chain variable region (VH) consists of the amino acid sequence SEQ ID NO:1 and amino acid sequences having at least 90% identity thereto.
64. Binding molecule according to any preceding item, which is an antibody construct.
65. Binding molecule according to any preceding item, which is selected from the group consisting of an antibody and an antigen binding fragment thereof, for example a Fab fragment, a Fab′ fragment, a F(ab′)₂ fragment, an Fc fragment, an Fv fragment, a single chain (scFv) fragment, an (scFv)₂ and a domain antibody.
66. Binding molecule according to any preceding item, which is an scFv.
67. Binding molecule according to any preceding item, further comprising a peptide linker joining the C terminus of said heavy chain variable region (VH) to the N terminus of said light chain variable region (VL).
68. Binding molecule according to item 67, wherein said peptide linker consists of the amino acid sequence SEQ ID NO:14.
69. Binding molecule according to any preceding item, which is capable of selective binding to a peptidic tag moiety comprised in a tag construct.
70. Binding molecule according to item 69, wherein the tag construct further comprises at least one cargo moiety.
71. Binding molecule according to item 70, wherein said at least one cargo moiety is an antigen moiety.
72. Binding molecule according to any one of items 70-71, wherein said at least one cargo moiety is a peptidic moiety or a nucleic acid moiety.
73. Binding molecule according to item 72, wherein the C terminus of said tag moiety is covalently bound to the N terminus of said at least one peptidic cargo moiety.
74. Binding molecule according to item 72, wherein the N terminus of said tag moiety is covalently bound to the C terminus of said at least one peptidic cargo moiety.
75. Binding molecule according to any one of items 71-74, wherein said at least one cargo moiety is a peptidic antigen moiety comprising a target antigen amino acid sequence.
76. Binding molecule according to any one of items 69-75, which is capable of binding to said tag moiety such that the K_{D} value of the interaction is at most 5×10⁻⁹ M, for example at most 1×10⁻¹⁰ M, for example at most 1×10⁻¹¹ M.
77. A bispecific conjugate comprising:
   at least one first moiety which is a binding molecule according to any one of the preceding items; and
   at least one second moiety which is an antibody or antigen binding fragment thereof.
78. Bispecific conjugate according to item 77, wherein said second moiety is an antigen binding fragment of an antibody selected from the group consisting of a Fab fragment, a Fab′ fragment, a F(ab′)₂ fragment, an Fc fragment, an Fv fragment, a single chain (scFv) fragment, an (scFv)₂ and a domain antibody.
79. Bispecific conjugate according to item 77, wherein said second moiety is an antibody, optionally of the IgG2 subtype.
80. Bispecific conjugate according to any one of items 77-79, wherein said first moiety is covalently linked to said second moiety.
81. Bispecific conjugate according to item 80, wherein said first moiety is covalently linked to said second moiety via a linker peptide.
82. Bispecific conjugate according to item 80, wherein said first moiety is linked to said second moiety directly.
83. Bispecific conjugate according to any one of items 80-82, wherein said first moiety is covalently linked to a C-terminal end of a light chain of said second moiety.
84. Bispecific conjugate according to any one of items 80-82, wherein said first moiety is covalently linked to an N-terminal end of a light chain of said second moiety.
85. Bispecific conjugate according to any one of items 80-82, wherein said first moiety is covalently linked to a C-terminal end of a heavy chain of said second moiety.
86. Bispecific conjugate according to any one of items 80-82, wherein said first moiety is covalently linked to an N-terminal end of a heavy chain of said second moiety.
87. Bispecific conjugate according to any one of items 77-86, wherein the conjugate comprises two first moieties and one antibody as second moiety, and one first moiety is conjugated to the CH3 domain of each heavy chain of said second moiety antibody.
88. Bispecific conjugate according to any one of items 77-86, wherein the conjugate comprises two first moieties and one antibody as second moiety, and one first moiety is conjugated to the CL domain of each light chain of said second moiety antibody.
89. Bispecific conjugate according to any one of items 77-88, wherein said second moiety is an anti-CD40 antibody or antigen binding fragment thereof.
90. Bispecific conjugate according to item 89, wherein said anti-CD40 antibody is selected from the group consisting of CP-870,893, APX005M, ADC-1013, ChiLob 7/4, SEA-CD40 and ABS-1150/1151 and an antibody comprising an antigen binding fragment derived from any one or more of said antibodies.
91. Bispecific conjugate according to item 89, wherein said anti-CD40 antibody or antigen binding fragment thereof comprises six complementarity determining domains (CDRs), wherein:
   VLCDR1 has the sequence set forth in SEQ ID NO:15;
   VLCDR2 has the sequence set forth in SEQ ID NO:16;
   VLCDR3 has the sequence set forth in SEQ ID NO:17;
   VHCDR1 has the sequence set forth in SEQ ID NO:18;
   VHCDR2 has the sequence set forth in SEQ ID NO:19; and
   VHCDR3 has the sequence set forth in SEQ ID NO:20.
92. Bispecific conjugate according to item 91, wherein
   the light chain variable domain of said anti-CD40 antibody comprises an amino acid sequence selected from SEQ ID NO:21 and amino acid sequences having at least 90% sequence identity thereto; and
   the heavy chain variable domain of said anti-CD40 antibody comprises an amino acid sequence selected from SEQ ID NO:22 and amino acid sequences having at least 90% sequence identity thereto.
93. Bispecific conjugate according to item 92, wherein said anti-CD40 antibody comprises
   a light chain comprising an amino acid sequence selected from SEQ ID NO:23 and amino acid sequences having at least 90% sequence identity thereto; and
   a heavy chain comprising an amino acid sequence selected from SEQ ID NO:24 and amino acid sequences having at least 90% sequence identity thereto.
94. A complex comprising:
   a bispecific conjugate according to any one of items 77-93; and
   a tag construct comprising a peptidic tag moiety.
95. Complex according to item 94, wherein said tag construct further comprises a cargo moiety, wherein said cargo moiety is a peptidic antigen moiety comprising a target antigen amino acid sequence, and wherein the C terminus of said tag moiety is covalently bound to the N terminus of said antigen moiety in said tag construct.
96. A polynucleotide encoding a binding molecule or conjugate according to any one of items 1-93.
97. An expression vector comprising a polynucleotide according to item 96.
98. A host cell comprising an expression vector according to item 97.
99. A method of producing a binding molecule or conjugate according to any one of items 1-93, the method comprising:
   culturing a host cell according to item 98 under conditions allowing expression of said binding molecule or conjugate from said vector; and
   isolating said binding molecule or conjugate.
100. A composition comprising a binding molecule according to any one of items 1-76 and at least one pharmaceutically acceptable excipient or carrier.
101. A composition comprising a conjugate according to any one of items 77-93 and at least one pharmaceutically acceptable excipient or carrier.
102. A composition comprising a complex according to any one of items 94-95 and at least one pharmaceutically acceptable excipient or carrier.
103. A kit comprising
   a conjugate according to any one of items 77-93;
   a peptidic tag construct comprising a peptidic tag moiety; and
   instructions to form a complex according to item 94.
104. Kit according to item 103, wherein said tag construct further comprises a cargo moiety, wherein said cargo moiety is a peptidic antigen moiety comprising a target antigen amino acid sequence, wherein the C terminus of said tag moiety is covalently bound to the N terminus of said antigen moiety in said tag construct, and wherein said instructions are instructions to form a complex according to item 95.
105. A binding molecule according to any one of items 1-76, a conjugate according to any one of items 77-93, a complex according to any one of items 94-95, a composition according to any one of items 100-102, or a kit according to any one of items 103-104, for use in therapy.
106. A binding molecule, conjugate, complex, composition or kit for use according to item 105 in the treatment or prevention of cancer, infection or an autoimmune disease.
107. A method of treatment or prevention of cancer, infection or an autoimmune disease comprising administering to a subject in need thereof of an effective amount of a binding molecule according to any one of items 1-76, a conjugate according to any one of items 77-93, a complex according to any one of items 94-95 or a composition according to any one of items 100-102.
108. Use of a binding molecule according to any one of items 1-76, a conjugate according to any one of items 77-93, a complex according to any one of items 94-95 or a composition according to any one of items 100-102 in the manufacture of a medicament.

1. A binding molecule, comprising:
an immunoglobulin heavy chain variable region (VH) consisting of an amino acid sequence selected from the group consisting of SEQ ID NO:1-6 and amino acid sequences having at least 90% identity thereto,
provided that said heavy chain variable region (VH) comprises three complementarity determining domains (CDRs), wherein:
VHCDR1 has the sequence set forth in SEQ ID NO:7;
VHCDR2 has the sequence IGRIDPEXₐX_{b}DAEYVP (SEQ ID NO:8), wherein XₐX_{b} is selected from the group consisting of SG, GG, QG, DG, NA and NG; and
VHCDR3 has the sequence set forth in SEQ ID NO:9; and
an immunoglobulin light chain variable region (VL) consisting of an amino acid sequence selected from the group consisting of SEQ ID NO:10 and amino acid sequences having at least 90% identity thereto,
provided that said light chain variable region (VL) comprises three complementarity determining domains (CDRs), wherein:
VLCDR1 has the sequence set forth in SEQ ID NO:11;
VLCDR2 has the sequence set forth in SEQ ID NO:12; and
VLCDR3 has the sequence set forth in SEQ ID NO:13.

2. Binding molecule according to claim 1, which is an scFv.

3. Binding molecule according to any preceding claim, which is capable of selective binding to a peptidic tag moiety comprised in a tag construct, said tag construct optionally further comprising at least one cargo moiety.

4. Binding molecule according to claim 3, wherein said cargo moiety is a peptidic antigen moiety comprising a target antigen amino acid sequence.

5. Binding molecule according to any of claims 3-4, which is capable of binding to said tag moiety such that the K_{D} value of the interaction is at most 5×10⁻⁹ M, for example at most 1×10⁻¹⁰ M, for example at most 1×10⁻¹¹ M.

6. A bispecific conjugate comprising:
at least one first moiety which is a binding molecule according to any one of the preceding claims; and
at least one second moiety which is an antibody or antigen binding fragment thereof, for example an anti-CD40 antibody or antigen binding fragment thereof.

7. Bispecific conjugate according to claim 6, wherein said second moiety is an anti-CD40 antibody or antigen binding fragment thereof comprising six complementarity determining domains (CDRs), wherein:
VLCDR1 has the sequence set forth in SEQ ID NO:15;
VLCDR2 has the sequence set forth in SEQ ID NO:16;
VLCDR3 has the sequence set forth in SEQ ID NO:17;
VHCDR1 has the sequence set forth in SEQ ID NO:18;
VHCDR2 has the sequence set forth in SEQ ID NO:19; and
VHCDR3 has the sequence set forth in SEQ ID NO:20.

8. Bispecific conjugate according to claim 7, wherein
the light chain variable domain of said anti-CD40 antibody comprises an amino acid sequence selected from SEQ ID NO:21 and amino acid sequences having at least 90% sequence identity thereto; and
the heavy chain variable domain of said anti-CD40 antibody comprises an amino acid sequence selected from SEQ ID NO:22 and amino acid sequences having at least 90% sequence identity thereto.

9. Bispecific conjugate according to any one of claims 7-8, wherein said anti-CD40 antibody comprises a light chain comprising an amino acid sequence selected from SEQ ID NO:23 and amino acid sequences having at least 90% sequence identity thereto; and
a heavy chain comprising an amino acid sequence selected from SEQ ID NO:24 and amino acid sequences having at least 90% sequence identity thereto.

10. A complex comprising:
a bispecific conjugate according to any one of claims 6-9; and
a tag construct comprising a peptidic tag moiety.

11. Complex according to claim 10, wherein said tag construct further comprises a cargo moiety, wherein said cargo moiety is a peptidic antigen moiety comprising a target antigen amino acid sequence, and wherein the C terminus of said tag moiety is covalently bound to the N terminus of said antigen moiety in said tag construct.

12. A polynucleotide or polynucleotides encoding a binding molecule or conjugate according to any one of claims 1-9.

13. A composition comprising a binding molecule according to any one of claims 1-5, a conjugate according to any one of claims 6-9 or a complex according to any one of claims 10-11, and at least one pharmaceutically acceptable excipient or carrier.

14. A kit comprising
a conjugate according to any one of claims 6-9;
a peptidic tag construct comprising a peptidic tag moiety; and
instructions to form a complex according to any one of claims 10-11.

15. A binding molecule according to any one of claims 1-5, a conjugate according to any one of claims 6-9, a complex according to any one of claims 10-11, a composition according to claim 13, or a kit according to claim 14, for use in therapy.

16. A binding molecule, conjugate, complex, composition or kit for use according to claim 15 in the treatment or prevention of cancer, infection or an autoimmune disease.
